(19) United States
(12) Patent Application Publication
(10) Pub. No.: US 2003/0032069 A1
(43) Pub. Date: Feb. 13, 2003

(54) Evaluating neuropshychiatric diseases using a specimen-linked database

(76) Inventor: Patrick J. Muraca, Pittsfield, MA (US)

Correspondence Address: PALMER & DODGE, LLP, PAULA CAMPBELL EVANS, 111 HUNTINGTON AVENUE, BOSTON, MA, 02199, US

(21) Appl. No.: 10/184,671

(22) Filed: Jun. 28, 2002

Related U.S. Application Data
(60) Provisional application No. 60/302,223, filed on Jun. 29, 2001.

(51) Int. Cl. G01N 33/567; G06F 19/00; G01N 33/48; G01N 33/50

(52) U.S. Cl. 435/7.21; 702/19

(57) ABSTRACT

The invention relates to a method and system for identifying and evaluating the responses of a patient to a neuropsychiatric disorder. Preferably, both physiological and behavioral responses are linked to molecular profiling data, i.e., data relating to the expression of a plurality of genes in tissues from the patient with these diseases. In one aspect, the invention provides a tissue information system comprising a specimen-linked database and an information management system for accessing, organizing, and displaying tissue information obtained from tissue microarrays comprising samples from patients with neuropsychiatric disorders.

### RELATED APPLICATIONS

This application claims priority under 35 U.S.C. §119(e) to U.S. Serial No. 60/302,223, filed Jun. 29, 2001. The entire teachings of the above application are incorporated herein by reference.

### FIELD OF THE INVENTION

The invention relates to microarrays comprising tissue samples from patients suffering from neuropsychiatric diseases and to a specimen-linked database for evaluating the same.

### BACKGROUND OF THE INVENTION

Research into the biochemical basis of behavior has identified a number of molecular pathways whose functions are likely to be critical in normal psychological functioning. For example, abnormalities in dopamine-based pathways have been implicated in schizoid behaviors (Blum et al., 1997, Mol. Psychiatry 2(3): 239-46), attention deficit hyperactivity disorder (ADHD) (Sunohara et al., 2000, J. Am. Acad. Child Adolesc. Psychiatry 39(12): 1537-42; Barr et al., 2000, Am. J. Med. Genet. 96(3): 262-7), conduct disorder or aggression (Comings et al., 2000, Clin. Genet. 58(1): 31-40), alcohol abuse (Blum et al, 1993, Alcohol 10(1): 59-67), stuttering, mania (Wu et al., 1997, Neuroreport. 8(3): 767-70; Nolan et al., 1983, J Affect Disord. 5(2): 91-6), sexual disorders (Hull et al., 1999, Behav. Brain. Res. 105(1): 105-16), and obsessive compulsive disorder (OCD) (see, e.g., Comings et al., 1996, Am. J Med. Genet. 67(3): 264-88).

Despite the association of dopamine-pathway genes (e.g., genes for dopamine synthesis, degradation, transporters, and receptors) with many neuropsychiatric disorders, the complexity of the dopamine pathway has made the development of diagnostic markers and drug targets for these diseases problematic. There are five known dopamine receptors (D1, D2, D3, D4, and D5), all of which are G Protein Coupled Receptors (GPCRs) which transmit signals to GTP-binding G proteins located on the inner surface of cell membranes. D1 and D5 form a “D1-like” receptor group and bind to G_{S} proteins, while D2, D3, and D4 receptors form a “D2-like” protein group and bind G₁ or G₀ proteins. Particular dopamine receptors have been associated with specific disorders. For example, the D2 receptor has been associated with ADHD, Tourette's Syndrome, conduct disorder, Post Traumatic Stress Syndrome and alcoholism (Comings et al, 1996, supra) and significant increases in dopamine D2 receptor density have been measured in individuals with detachment social isolation and lack of intimate friendships (Farde et al, 1997, Nature, 385(6617): 590). Mutations in D4, in contrast, are associated with schizophrenia (see, e.g., U.S. Pat. No. 6,203,998).

Still other studies have implicated the involvement of combinations of dopamine receptors in certain neuropsychiatric disorders, such as substance abuse disorders (see, e.g., Comings et al., 1999, Moll. Psychiatry 4(5): 484-7) where D2 and D3 receptors have both been implicated and in the psychoses experienced by some Alzheimer's patients (see, e.g., Sweet et al., 1998, Arch Neural. 55(10): 1335-40) where an involvement of D1, D2, and D3 has been shown. Additionally, because dopamine pathway genes interact with other signaling pathways such as the serotonin, norepinephrin, GABA, opioid, and cannabinoid pathways, defects in one or more genes in any of these pathways can produce similar symptoms (see, e.g., Comings et al., 2000, Prog. Brain Res. 126: 325-41). For example, schizophrenia has been associated with biochemical abnormalities in the dopamine, GABA, glutamate, NMDA, and nicotinic receptor systems (see, e.g., Pearlson et al., 2000, Ann. Neurol. 48(4): 556-66). These studies demonstrate that neuropsychiatric disorders are generally complex polygenic disorders with variable penetrance and environmental components (Lander and Schork, 1994, Science 265(5181): 2037-48).

The sequencing of the human genome and the advance of high throughput techniques has made it possible to evaluate the expression of multiple RNA transcripts and polypeptides at a time, making it more feasible to apply a genome-wide or proteome-wide approach to the study of neuropsychiatric disorders. For example, John-ston-Wilson et al., 2000, Molecular Psychiatry 5: 142-149, report using a proteomic approach to compare over 200 proteins expressed in a large number of samples from schizophrenics, identifying at least 8 proteins whose expression is altered in these patients. However, while these techniques readily identify differentially expressed genes, the generation of systematic approaches to analyze the role these genes play in physiological responses have lagged behind.

The use of “computational biology” or “bioinformatics” to solve biological data analysis problems has developed as a way to address this problem and database systems for gene expression monitoring have been described in the art. U.S. Pat. No. 6,185,561 describes a database model to facilitate molecular profiling or “data mining” of expression information from nucleic acid arrays. However, the patent does not describe how to model interactions between the products of expressed genes.

Genomic and proteomic information relating to GPCRs, including neurotransmitters such as dopamine, have been collected and organized in a web-based system, the GPCRDB Information System, which can be accessed through the World Wide Web using the URL http://www.gpcr.org/7tm/. The GPCRDB system includes links to genomic databases, protein databases, drug databases, and various reference databases. The system includes sequence information, mutant data, and ligand binding constant information and provides computational alignment tools, three-dimensional models, phylogenetic trees and two dimensional visualization tools. However, the system does not link the various databases to clinical information.

International Application WO 99/44062 describes methods for rapid molecular profiling of tissues or other cellular specimens. The publication describes correlating data obtained from tissue microarrays with clinical information from patients and suggests the use of a database for analyzing and correlating different molecular characteristics of tissue samples. The publication does not describe how to use such a database to identify interactions between multiple gene products.

U.S. Pat. No. 5,980,096 describes a computer-based system for modeling and simulating complex systems, but does not evaluate patient characteristics in this process.

### SUMMARY OF THE INVENTION

In one aspect, the invention provides an information system, comprising a specimen-linked database comprising information about at least one microarray identified by an identifier, the microarray comprising one or more tissue or cell samples from at least one patient with a neuropsychiatric disorder. Preferably, the system also comprises at least one user device connectable to the network, for displaying an interface onto which a user can input the identifier, enabling the user to access the database. The tissue microarray generally comprises a plurality of sublocations, each sublocation identifiable by coordinates. In one aspect, after the user has inputted the identifier onto the interface displayed by the user device, the system displays another interface which provides a plurality of selectable coordinates corresponding to the coordinates on said tissue microarray. Selection of a coordinate causes the system to display information about a tissue sample at the sublocation identified by the coordinates. Preferably, each coordinate is associated with a link for linking a user to the database.

In one aspect, when a user selects the link, an interface providing information categories is displayed, each information category associated with a link to a portion of the database comprising information relating to the information category. In another aspect, after the user has inputted the identifier, the system displays an interface on the display of the user device which presents a representation of the tissue microarray. Preferably, the representation comprises images of tissues at different sublocations on the microarray. In one aspect, each image is associated with a link for linking a user to the database. In another aspect, after a user inputs the identifier, an interface is displayed on the user device which comprises one or more fields for inputting coordinates of a sublocation of a tissue microarray about which the user desires access to information about. Preferably, after this inputting, the system displays an interface providing information categories relating to information available about a tissue sample at said sublocation.

In a preferred aspect, the specimen-linked database comprises records relating to the physiological responses of a plurality of patients having neuropsychiatric disorders. The records preferably comprise gene expression data. Preferably, this data comprises data relating to the expression of a plurality of pathway biomolecules. For example, the pathway biomolecules can comprise neurotransmitter receptor signaling molecules. In one aspect, the neurotransmitter receptor is selected from the group consisting of an adrenoreceptor, a dopamine receptor, an opioid receptor, cannabinoid receptor, a muscarinic receptor, an NMDA receptor, an mGlu receptor, a GABA receptor, a serotonin receptor, and combinations thereof. In another aspect, the pathway comprises a neurotransmitter, a neurotransmitter receptor, biomolecules involved in neurotransmitter synthesis, a neurotransmitter transporter, a G protein, and a kinase. Preferably, information relating to samples on the microarray is indexed in the database using one or more of SNOWMED codes, DSM-IV-TR codes, and ICD-9 codes.

In one aspect, the neuropsychiatric disorder is classified using DSM-IV criteria and preferably, records in the specimen-linked database are indexed according to the DSM-IV classification of patients providing the information in these records. Information can be obtained from one or more autopsy procedures and/or from living patients. In another aspect, the information system comprises records relating to the behavioral responses of a plurality of patients having neuropsychiatric disorders. These behavioral responses can include responses to a questionnaire and/or can be obtained from records of psychological evaluations of patients by health care workers. The specimen-linked database preferably also comprises patient information (e.g., information relating to age, sex, medical history, family medical history, exposure to drugs, and the like).

In one aspect, accessing the database provides information relating to one or more of diagnosis and treatment.

In another aspect, the invention provides a method for obtaining information relating to physiological responses of a patient suspected of having a neuropsychiatric disorder, comprising: providing a user with a microarray comprising tissues or cells from the patient, providing the user with an identifier which identifies the microarray, providing the user with access to the system described above and displaying the interface onto which the user can input the identifier, and allowing the user to input the identifier, wherein the system, in response to this inputting displays an interface providing information relating to the microarray identified by the identifier. Preferably, the system comprises an information management system comprising search and relationship determining functions.

In one aspect, in response to inputting by the user, the system displays a new information interface comprising one or more fields into which a user can input information relating to the microarray. New information can include information relating to the expression of one or more neurotransmitter receptor pathway biomolecules in samples on the microarray and/or patient information about patients who supplied the samples. In one aspect, the new information relates to behavioral responses of the patient. In another aspect, the new information is information relating to the expression of one or more neurally expressed genes in samples on said microarray. The new information can also relate to the expression of one or more EST sequences in samples on the microarray.

In one aspect, expression is determined by reacting the microarray with a molecular probe which specifically binds to a biomolecule; for example, the probe can be a nucleic acid, an aptamer, an antibody, or combinations thereof.

Preferably, the system used in the method further comprises an information management system comprising search and relationship determining functions and after inputting an identifier identifying a microarray being evaluated for expression of one or more biomolecules, the information management system implements its relationship determining function to identify any relationship between the expression of the one or more biomolecules and the neuropsychiatric disease. In one aspect, the relationship identified is used to provide a diagnosis and/or treatment options to the patient.

In one aspect, the invention also provides a method for identifying a molecular marker of a neuropsychiatric disorder. The method comprises the steps of: providing a microarray comprising neural samples from first patients having a neuropsychiatric disorder, the patients being diagnosed using a first classification system (e.g., such as DSM-IV), providing neural samples from second patients on the same or a different microarray, the second patients not having the disorder but, preferably, sharing similar demographic characteristics as the first patients, providing non-neural samples from third patients having the neuropsychiatric disorder, the third patients being diagnosed using the same classification system and, preferably, having similar demongraphic characteristics as the first patients, and providing non-neural samples from fourth patients without the disorder, the fourth patients, preferably, having similar demographic characteristics as the first patients. The microarrays and non-neural samples are reacted with a molecular probe which specifically binds to a biomolecule expressed in neural cells and the reactivity of the molecular probe with samples in the microarrays and the non-neural samples is determined. A biomolecule is identified as a marker biomolecule if the biomolecule is differentially expressed in neural samples from patients having the neuropsychiatric disorder compared to samples from patients without the disorder and is also differentially expressed in the non-neural samples.

Preferably, the neural samples from the first and second patients are obtained from autopsies while the non-neural samples are obtained from living patients. Preferably, non-neural samples are obtained from bodily fluids. Like the neural samples, the non-neural samples can be arrayed on a substrate, thereby forming a microarray. In one aspect, microarrays used in the method are identified by identifiers and information relating to the expression of the biomolecule is stored in the specimen-linked database described above. The method provides a way to identify markers of neurological disease assayable in accessible tissues from the body of a living patient.

In another aspect, the invention provides a microarray comprising a plurality of tissue or cell samples, at least one of the samples being from a patient with a neuropsychiatric disorder. The microarray is preferably identified by an identifier and information relating to samples on the microarray is stored within the system described above, and is accessible to a user when the user enters the identifier into an interface displayed by a user device of the system.

In still another aspect, the invention provides a microarray comprising a plurality of tissue or cell samples, at least one of said samples being from a patient with a neuropsychiatric disorder, wherein at least one of the samples is frozen.

The invention further provides a method for obtaining information about a sample in a microarray. The microarray comprises a plurality of samples, at least one of the samples being from a patient with a neuropsychiatric disorder. The method comprises the steps of: providing an interface on a display of a user device connectable to the network, displaying a plurality of selectable coordinates on the interface, each coordinate representing one of the samples in the microarray and each coordinate associated with a link for accessing a database, the database comprising information relating to the one of the samples in the microarray; and allowing a user to select a link associated with one of the coordinates, to thereby access the database and obtain information about the sample.

### BRIEF DESCRIPTION OF THE DRAWINGS

The objects and features of the invention can be better understood with reference to the following detailed description and accompanying drawings.

FIG. 1A shows a tissue microarray according to the present invention comprising a plurality of sublocations, each sublocation comprising a tissue sample whose morphological features can be distinguished under a microscope. FIG. 1B shows a profile array substrate comprising a first location for a test sample and a second location comprising a tissue micro array.

FIGS. 2A-2C show an interface on a display of a user device connectable to a network which displays information relating to the biological characteristics of tissues at different sublocations in a tissue microarray. FIG. 2A shows an interface for addressing a neuropsychiatric disease microarray and for inputting new information relating to the tissue samples in the microarray into a database. FIG. 2B shows a display of a portion of the database. FIG. 2C shows a display on the interface of the device which displays relationships identified between psychiatric data and molecular profiles obtained for tissue samples on the tissue microarray.

FIG. 3 is a schematic diagram illustrating a system comprising a specimen-linked database and information management system according to one aspect of the invention.

FIG. 4 shows an exemplary data table obtained using the system of the invention, in which information about tissue specimens is cross-referenced to the database using ICD-9-CM and DSM-IV-TR codes, in one aspect of the invention.

### DETAILED DESCRIPTION

The invention relates to a method and system for identifying and evaluating the responses of a patient to a neuropsychiatric disorder. Preferably, both physiological and behavioral responses are linked to molecular profiling data, i.e., data relating to the expression of a plurality of genes in tissues from the patient with these diseases. In one aspect, the invention provides a tissue information system comprising a specimen-linked database and an information management system for accessing, organizing, and displaying tissue information obtained from tissue microarrays comprising samples from patients with neuropsychiatric disorders. Definitions The following definitions are provided for specific terms which are used in the following written description.

As used herein, the term “information about the patient” refers to any information known about an individual (a human or non-human animal) from whom a tissue sample was obtained. The term “patient” does not necessarily imply that the individual has ever been hospitalized or received medical treatment prior to obtaining a tissue sample. The term “patient information” includes, but is not limited to, age, sex, weight, height, ethnic background, occupation, environment, police records, family medical background, the patient's own medical history (e.g., information pertaining to prior diseases, diagnostic and prognostic test results, DSM-IV-TR classification, psychological evaluations, drug exposure or exposure to other therapeutic agents, responses to drug exposure or exposure to other therapeutic agents, results of treatment regimens, their success, or failure, history of alcoholism, drug or tobacco use, cause of death, and the like). The term “patient information” refers to information about a single individual. Information from multiple patients provides “demographic information,” defined as statistical information relating to populations of patients, organized by geographic area or other selection criteria, while “epidemiological information” is defined as information relating to the incidence of disease in populations.

As used herein, the “similar demographic characteristics” or “demographically matched”, refers to patients who minimally share the same sex and belong to the same age grouping (e.g., are within about 5 to fifteen years of a selected age). Additional shared characteristics can be selected including, but not limited to, shared place of residence (e.g., within a hundred mile radius of a particular location), shared occupation, shared history of illnesses, and the like.

As defined herein, the term “information relating to” is information which summarizes, reports, provides an account of, and/or communicates particular facts, and in some embodiments, includes information as to how facts were obtained and/or analyzed.

As used herein, the term, “in communication with” refers to the ability of a system or component of a system to receive input data from another system or component of a system and to provide an output in response to the input data. “Output” may be in the form of data or may be in the form of an action taken by the system or component of the system.

As used herein, the term “provide” means to furnish, supply, or to make available.

As defined herein, a “tissue” is an aggregate of cells that perform a particular function in an organism. The term “tissue” as used herein refers to cellular material from a particular physiological region. The cells in a particular tissue may comprise several different cell types. A non-limiting example of this would be brain tissue that further comprises neurons and glial cells, as well as capillary endothelial cells and blood cells. The term “tissue” also is intended to encompass a plurality of cells contained in a sublocation on the tissue microarray that may normally exist as independent or non-adherent cells in the organism, for example immune cells, or blood cells. The term is further intended to encompass cell lines and other sources of cellular material which represent specific tissue types e.g., by virtue of expression of biomolecules characteristic of specific tissue types).

As defined herein, a “molecular probe” is any detectable molecule, or is a molecule which produces a detectable molecule upon reacting with a biological molecule. “Reacting” encompasses binding, labeling, or catalyzing an enzymatic reaction. A “biological molecule” or “biomolecule” is any molecule which is found in a cell or within the body of an organism.

As used herein, the term “biological characteristics of a tissue” refers to the phenotype and genotype of the tissue or cells within a tissue, and includes tissue type, morphological features; the expression of biological molecules within the tissue (e.g., such as the expression and accumulation of RNA sequences, the expression and accumulation of proteins (including the expression of their modified, cleaved, or processed forms (active or inactive), and further including the expression and accumulation of enzymes, their substrates, products, and intermediates); and the expression and accumulation of metabolites, carbohydrates, lipids, and the like). A biological characteristic can also be the ability of a tissue to bind, incorporate, or respond to a drug or agent. “Biological characteristics of a tissue source” are the characteristics of the organism which is the source of the tissue (e.g., such as the age, sex, and physiological state of the organism) and encompasses patient information.

As defined herein, “a diagnostic trait” is an identifying characteristic, or set of characteristics, which in totality, are diagnostic. The term “trait” encompasses both biological characteristics and experiences (e.g., exposure to a drug, occupation, place of residence). In one embodiment, a trait is a marker for a particular cell type, such as a transformed, immortalized, pre-cancerous, or cancerous cell, or a state (e.g., a disease) and detection of the trait provides a reliable indicia that the sample comprises that cell type or state. Screening for an agent affecting a trait thus refers to identifying an agent which can cause a detectable change or response in that trait which is statistically significant within 95% confidence levels.

As used herein, the term “expression” refers to a level, form (which may be active or inactive), or localization of a product. For example, “expression of a protein” refers to any or all of the level, form (e.g., presence, absence, or quantity of modifications, or cleavage or other processed products or allosteric conformations), or localization (e.g., subcellular and/or extracellular compartment) of the protein.

A “disease or pathology” is a change in one or more biological characteristics that impairs normal functioning of a cell, tissue, and/or organism. A “pathological condition” encompasses a disease but also encompasses abnormal responses which are not associated with any particular infectious organism or single genetic alteration in an individual. For example, as defined herein, a stroke or an immune response occurring after transplantation of an organism would be encompassed by the term “pathological condition.”

As used herein, the term “difference in biological characteristics” refers to an increase or decrease in a measurable expression of a given biological characteristic. A difference may be an increase or a decrease in a quantitative measure (e.g., amount of a protein or RNA encoding the protein) or a change in a qualitative measure (e.g., location of the protein). Where a difference is observed in a quantitative measure, the difference according to the invention will be at least about 10% greater or less than the level in a normal standard sample. Where a difference is an increase, the increase may be as much as about 20%, 30%, 50%, 70%, 90%, 100% (2-fold) or more, up to and including about 5-fold, 10-fold, 20-fold, 50-fold or more. Where a difference is a decrease, the decrease may be as much as about 20%, 30%, 50%, 70%, 90%, 95%, 98%, 99% or even up to and including 100% (no specific protein or RNA present). It should be noted that even qualitative differences may be represented in quantitative terms if desired. For example, a change in the intracellular localization of a polypeptide may be represented as a change in the percentage of cells showing the original localization.

As defined herein, the “efficacy of a drug” or the “efficacy of a therapeutic agent” is defined as ability of the drug or therapeutic agent to restore the expression of diagnostic trait to values not significantly different from normal (as determined by routine statistical methods, to within 95% confidence levels).

As defined herein, “a tissue microarray” is a microarray that comprises a plurality of sublocations, each sublocation comprising tissue cells and/or extracellular materials from tissues, or cells typically infiltrating tissues, where the morphological features of the cells or extracellular materials at each sublocation are visible through microscopic examination. The term “microarray” implies no upper limit on the size of the tissue sample on the array, but merely encompasses a plurality of tissue samples which, in one embodiment, can be viewed using a microscope.

As defined herein, “a whole body microarray” is a microarray comprising tissue and/or cell samples representing the whole body of an organism. In one embodiment, the microarray comprises at least about five different tissue samples from an organism, at least about ten different tissues from an organism, or at least about 20 different tissues from an organism. For example, in one embodiment, a whole body microarray comprises at least about five different tissues selected from the group consisting of brain tissue, cardiac tissue, liver tissue, pancreatic tissue, spleen tissue, stomach tissue, lung tissue, skin tissue, eye tissue, colon tissue, reproductive organ tissue, and kidney tissue. In preferred embodiments, a sample of a bodily fluid is also included, such as a blood sample (whole blood, serum, or plasma), lymph sample, and the like.

As defined herein a “a sample” is a material suspected of comprising an analyte and includes a biological fluid, suspension, buffer, collection of cells, scraping, fragment or slice of tissue. A biological fluid includes blood, plasma, sputum, urine, cerebrospinal fluid (CSF), lavages, and leukophoresis samples.

The term “donor block” as used herein, refers to tissue embedded in an embedding matrix, from which a tissue sample can be obtained and placed directly onto a slide or placed into a receptacle of a recipient block.

The term “recipient block” as used herein, refers to a block formed from an embedding matrix, having which comprises a plurality of tissue samples; each tissue sample forming the source of a sublocation on a tissue microarray. The relative positions of tissue samples are maintained when the recipient block is sectioned, such that each section comprises sublocations at identical coordinates as any other section from the recipient block.

As defined herein, a “nucleic acid microarray,” a “peptide microarray” or “small molecule” microarray refers to a plurality of nucleic acids, peptides, or small molecules, respectively, respectively that are immobilized on a substrate in assigned (i.e., known) locations on the substrate.

As defined herein, a “database” is a collection of information or facts organized according to a data model which determines whether the data is ordered using linked files, hierarchically, according to relational tables, or according to some other model determined by the system operator. The organization scheme that the database uses is not critical to performing the invention, so long as information within the database is accessible to the user through an information management system. Data in the database are stored in a format consistent with an interpretation based on definitions established by the system operator (i.e., the system operator determines the fields which are used to define patient information, molecular profiling information, or another type of information category). As used herein, a “specimen-linked database” is a database which cross-references information in the database to tissue specimens provided on one or more microarrays, and preferably using codes, such as SNOMED® codes, ICD-9 codes, and/or DSM-IV TR codes. As used herein a “subdatabase” is a portion of a database in which records of a particular type are stored.

As defined herein, “a system operator” is an individual who controls access to the database.

As used herein, the term “information management system” refers to a system which comprises a plurality of functions for accessing and managing information within the database. Minimally, an information management system according to the invention comprises a search function, for locating information within the database and for displaying a least a portion of this information to a user, and a relationship determining function, for identifying relationships between information or facts stored in the database.

As defined herein, an “interface” or “user interface” or “graphical user interface” is a display (comprising text and/or graphical information) displayed by the screen or monitor of a user device connectable to the network which enables a user to interact with the database and information management system according to the invention.

As used herein, the term “link” refers to a point-and-click mechanism implemented on a user device connectable to the network which allows a viewer to link (or jump) from one display or interface where information is referred to (“a link source”), to other screen displays where more information exists (a “link destination”). The term “link” encompasses both the display element that indicates that the information is available and a program which finds the information (e.g., within the database) and displays it one the destination screen. In one embodiment, a link is associated with text; however, in other embodiments, links are associated with images or icons. In some embodiments, selecting a link (e.g., by right clicking using a mouse) will cause a drop down menu to be displayed which provides a user with the option of viewing one of several interfaces. Links can also be provided in the form of action buttons, radiobuttons, check buttons and the like.

As defined herein, a “browser” is a program which supports the displaying of documents, across a network. Browsers enable accessing linked information over the Internet and other networks, as well as from magnetic disk, CD-ROM, or other memory sources.

The term “providing access to at least a portion of a database” as defined herein refers to making information in the database available to user(s) through a visual or auditory means of communication.

As used herein, “through a visual means of communication” includes displaying or providing written text, image(s), or a combination of written and graphical information to a user of the database.

As used herein, “through an auditory means of communication” refers to providing the user with taped audio information, or access to another user who can communication the information through speech or sign language. Written and/or graphical information can be communicated through a printed report or electronically (e.g., through a display on the display of a computer or other processor, through email or other electronic messaging systems, through a wireless communications device, via facsimile, and the like). Access can be unrestricted or restricted to specific subdatabases within the database.

As used herein, “pathway molecules” or “pathway biomolecules” are molecules involved in the same pathway and whose accumulation and/or activity and/or form (i.e., referred to collectively as the “expression” of a molecule) is dependent on other pathway molecules, or whose accumulation and/or activity and/or form affects the accumulation and/or activity or form of other pathway target molecules. For example, a “neurotransmitter receptor pathway molecule” is a molecule whose expression is affected by the interaction of a neurotransmitter receptor(e.g., such as a dopamine receptor) and its cognate ligand (e.g., such as dopamine). Thus, a neurotransmitter receptor itself is a neurotransmitter receptor pathway molecule, as is its ligand, as are second messenger molecules which are activated or inhibited when the receptor binds to its ligand. An “early pathway molecule” is a molecule whose expression is required for the expression of at least about five other genes, while a “late pathway” molecule is a molecule whose expression or activation is required for the expression or activation of about two or fewer other genes. Pathways can be further divided into subpathways; thus, a dopamine pathway can be subdivided into a D1 pathway, a D2 pathway, a D3 pathway, a D4 pathway, and a D5 pathway based on the types of dopamine receptors being evaluated. Pathway molecules can also include gene products involved in synthesis, degradation, transport (e.g., uptake) of other molecules in the pathway.

As used herein, a “physiological response” refers to a change in one or more functions of a cell, tissue, organ, or a plurality of the foregoing in the body of an organism.

Additional definitions may be found in U.S. patent application Ser. No. 09/781,016 “Specimen-Linked Database” filed Feb. 9, 2001, the entirety of which is incorporated by reference herein.

Microarrays

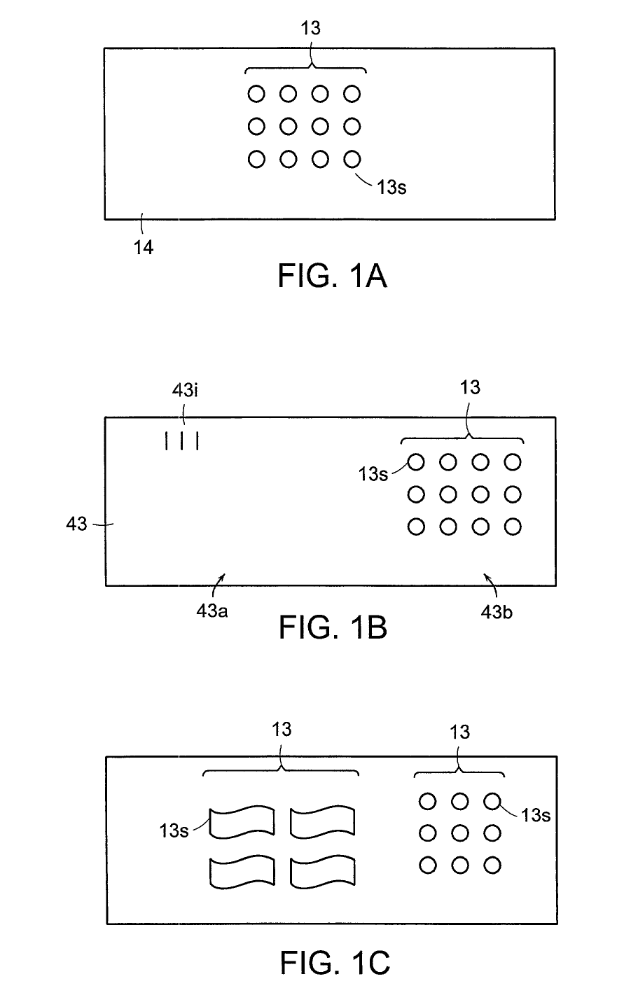

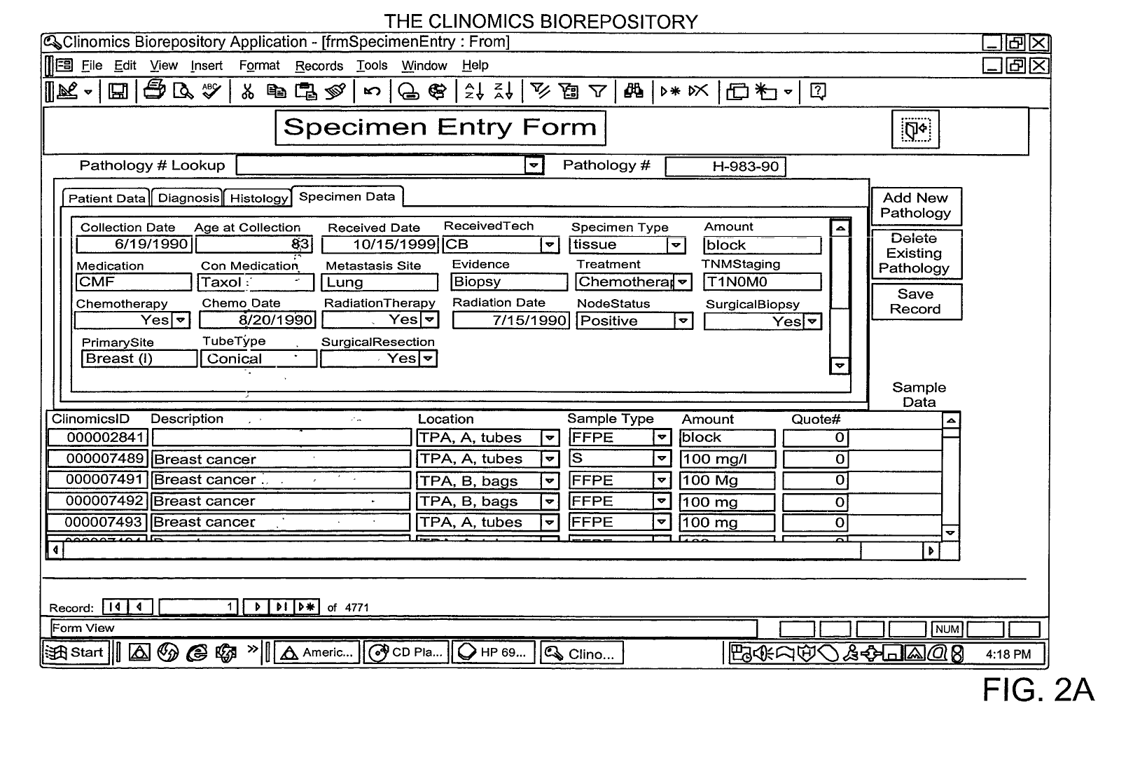

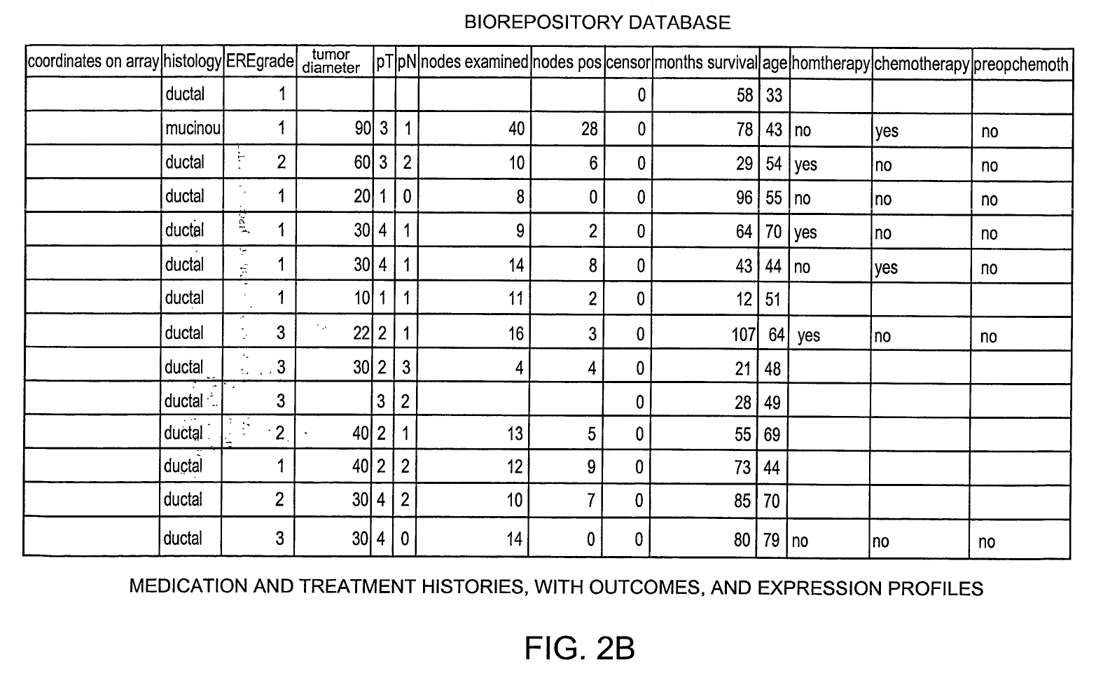

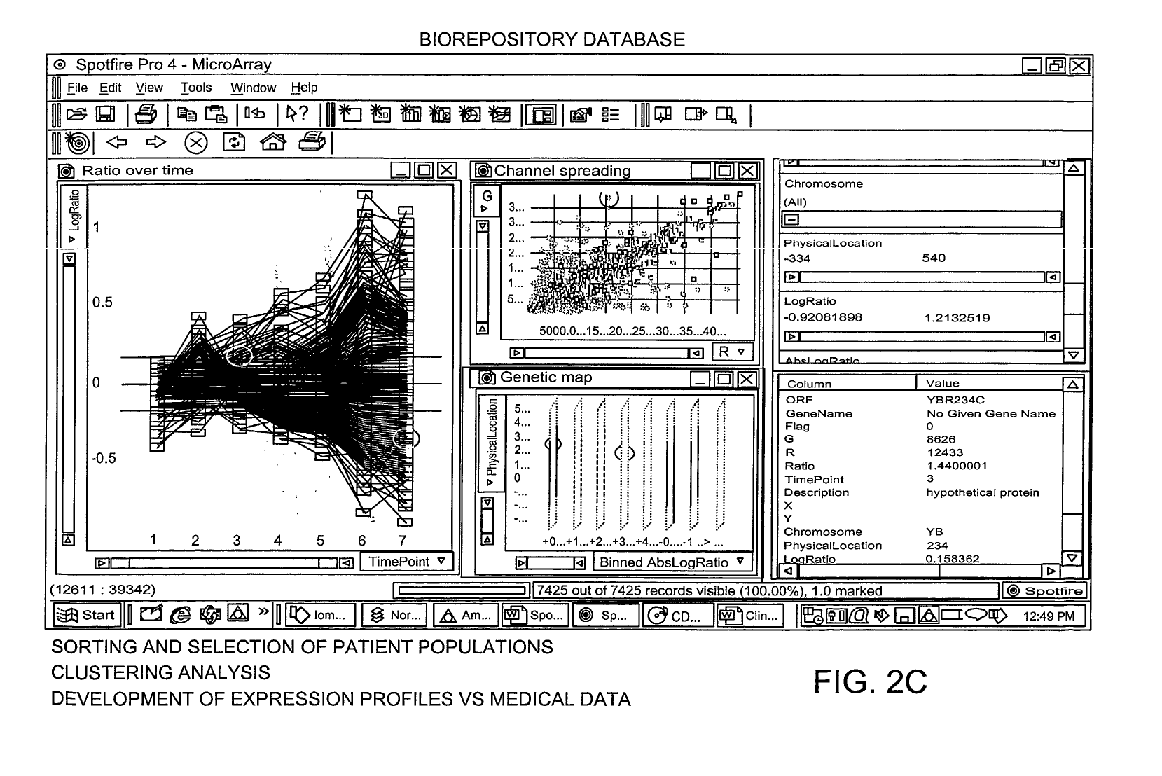

As shown in FIG. 1A, microarrays 13 according to the invention comprise a plurality of sublocations 13s, each sublocation comprising a tissue/cell sample having at least one known biological characteristic (e.g., such as tissue type). In one embodiment, the sample at at least one sublocation 13s has substantially intact morphological features which at least can be viewed under a microscope to distinguish subcellular features (e.g., such as a nucleus, an intact cell membrane, organelles, and/or other cytological features), i.e., the sample is not lysed.

In one aspect of the invention, the microarray comprises a substrate 43 to facilitate handling of the microarray 13 through a variety of molecular procedures. As used herein, “molecular procedure” refers to contact with a test reagent or molecular probe such as an antibody, nucleic acid probe, enzyme, chromagen, label, and the like. In one embodiment, a molecular procedure comprises a plurality of hybridizations, incubations, fixation steps, changes of temperature (from −4° C. to 100° C.), exposures to solvents, and/or wash steps. Suitable substrates are described in U.S. patent application Ser. No. 09/781,016 “Specimen-Linked Database” filed Feb. 9, 2001.

In another aspect, the substrate 43 is designed to accommodate a control microarray (e.g., comprising samples whose reactivity with at least one molecular probe is known) and a test tissue or cell sample for comparison with the control microarray. As shown in FIG. 1B, such a “profile microarray e substrate” 43 comprises a first location 43a for placing a test sample and a second sublocation 43b comprising the microarray 13. The profile microarray substrate 43 allows testing of a test tissue sample to be done simultaneously with the testing of samples on the microarray 13. This enables a side-by-side comparison of biological characteristics expressed in the test sample with the characteristics of the tissues/cells in the microarray 13. Profile microarray substrates 43 are disclosed in U.S. Provisional Application Serial No. 60/234,493, filed Sep. 22, 2000, the entirety of which is incorporated by reference herein.

Sources of Tissue

Tissue samples can be obtained as sections, slices, or fragments of tissues or can be obtained from suspensions of cells obtained from tissues (e.g., a suspension of minced brain cells, spinal cord tissue, and the like). Cells also can also be obtained from mucosal tissues, e.g., from nasal swabs, buccal scrapings, or pap smears, as well as from bodily fluids, for example, plasma, serum, saliva, and the like, or from procedures such as bronchial lavages, amniocentesis procedures or leukophoresis. In some aspects, cells are cultured first prior to being embedded to expand a population of cells being analyzed. Cells from continuously growing cell lines can also be used as well as cells which are purified (e.g., flow sorted, or collected by density gradient centrifugation to be enriched for one cell type).

Tissues at individual sublocations 13s can be obtained from cadavers or patients who have recently died (e.g., from autopsies), and/or from surgical specimens, pathology specimens, from “clinical waste” tissue that would normally be discarded from other procedures.

Preferably, the microarray 13 comprises at least one neural tissue sample, such as a brain tissue sample and/or spinal cord tissue sample. These are generally obtained from autopsies or surgical and other pathology procedures (e.g., biopsies, and the like). In one aspect, the microarray 13 comprises tissues representative of the whole body of a patient (e.g., tissues from at least about five different organs, and preferably at least about ten different organs from a patient).

Preferably, these patients represent individuals who have been diagnosed using DSM-IV-TR criteria as having one or more neuropsychiatric disorders. Neuropsychiatric disorders encompassed within the scope of the invention include, but are not limited to, mental retardation, a learning disorder, a motor skills disorder, a communication disorder, a pervasive developmental disorder (e.g., autism, childhood disintegrative disorder, Rett's disorder), attention deficit and disruptive behavior disorders, eating disorders, tic disorders, elimination disorders (encopresis, enurisis), selective mutism, separation anxiety disorder, reactive attachment disorder of infancy or early childhood, delirium, dementia, amnestic disorders, cognitive disorders, catatonic disorder, personality change disorder, substance dependence or other substance induced disorders (e.g., a drug or alcohol abuse related disorder), schizophrenia (e.g., catatonic, disorganized, paranoid, residual, undifferentiated), schizophreniform disorder, delusional disorder, brief psychotic disorder, shared psychotic disorder, psychotic disorder due to a general medical condition (e.g., delusions, hallucinations), a substance-induced psychotic disorder, mood episodes (major depressive episode, hypomanic episode, manic episode, mixed episode), depressive disorders, bipolar disorders, acute stress disorder, agoraphobia, anxiety disorder, obsessive-compulsive disorder, panic disorder with or without agoraphobia, postraumatic stress disorder, obsessive-compulsive disorder, body dysmorphic disorder, conversion disorder, hypochondriasis, and other somatoform disorders, a dissociative disorder, a sexual or gender identity disorder, an eating disorder (e.g., anorexia, bulimia nervosa), a sleep disorder, kleptomania, pyromania, pathological gambling, intermittent explosive disorder, and an Axis II personality disorder (each disorder as classified using DSM-IV criteria). In some aspects, tissues are obtained from patients with a plurality of disorders.

In one aspect, sets of microarrays 13 are provided representing multiple individuals with tissue specimens covering at least about 5, 10, 15, 20, 25, 30, 40, or at least about 50 different disease categories, including, but not limited to, one or more of the DSM-IV categories identified above.

In one aspect, because of the desirability of evaluating samples from patients receiving ongoing psychiatric treatment, samples are obtained from bodily fluids or accessible cells (e.g., from nasal or buccal swabs) of living patients. As discussed in Chelly et al., 1989, Proc. Natl. Acad. Sci. USA 86(8): 2617-21 and U.S. Pat. No. 5,962,664, the entireties of which are incorporated by reference herein, gene expression in accessible tissues where a gene product does not have a direct impact on function can still serve to monitor gene function/physiological responses in inaccessible tissues where these genes do function.

In some aspects, microarrays are provided which comprise tissue samples from patients suffering from a neurodegenerative disease who additionally have also been diagnosed with a mood disorder or psychosis. Neurodegenerative diseases encompassed within the scope of the invention encompass chronic neurodegenerative diseases, including, but not limited to: AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supra-nucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi-system disorder); demyelinating core disorders, such as multiple sclerosis, acute transverse myelitis; and disorders of the motor unit such as neurogenic muscular atrophies (anterior horn cell degeneration, such as amyotrophic lateral sclerosis, primary lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis Hallerrorden-Spatz disease; and Dementia pugilistica, diabetic peripheral neuropathy. (see, e.g., Berkow et al, eds., The Merck Manual, 16th edition, Merck and Co., Rahway, N.J., 1992, which reference, and references cited therein, are entirely incorporated herein by reference). Acute neurodegenerative diseases are also encompassed within the scope of the invention, such as conditions arising from stroke, cerebral ischemia resulting from surgery and epilepsy, as well as hypoglycemia and trauma resulting in injury of the brain, peripheral nerves or spinal cord, and the like.

The microarray 13 can comprise tissue samples from one or more patients who have been exposed to a drug or agent (e.g., a toxin) or an environmental condition in addition to having a neuropsychiatric disorder. The patient also may have one or more underlying and/or concurrent diseases or pathological conditions. In one aspect, tissue samples are obtained from a plurality of patients having neuropsychiatric disorders who share the same demographic characteristics (e.g., same age, gender, underlying disease conditions) but who have been exposed to different doses of a drug or agent. In another aspect, samples are obtained from demographically matched patients who have been exposed for varying periods of time to a drug or agent or environmental condition.

It is contemplated that for all of the above scenarios, tissues/cells (“control donor samples”) are also obtained from normal patients or from patients not having a neuropsychiatric disease but who are otherwise demographically matched with patients having neuropsychiatric diseases who are supplying donor samples (“test donor samples”) for the microarrays (e.g., sharing the same underlying illnesses, and other characteristics such as age, sex, and the like), thereby providing control samples for the microarrays 13. Control donor samples can be provided on the same microarray as test donor samples or can be provided on separate microarrays.

Although in a preferred embodiment of the invention, the microarrays 13 comprise human tissues and/or cell samples, in one aspect of the invention, tissues from other organisms are arrayed. For example, the microarray 13 can comprise tissues from non-human animals which provide a model of a neuropsychiatric disorder or an aberrant behavioral response (e.g., such as high levels of aggression). The microarray 13 preferably comprises multiple tissues from such a non-human animal. In some aspects, the animals providing donor samples have been exposed to a therapeutic agent for treating the disorder (e.g., drugs, antibodies, proteins, genes, antisense molecules, ribozyymes, aptamers, combinations thereof, and the like). Thus, the microarrays 13 can be used to examine dose responses of therapeutic agents in animal models of neuropsychiatric disorders and the distribution of the therapeutic agent in multiple tissues, in addition to neural tissues, at different time points can be examined using these arrays. Examples of non-human animal models of neuropsychiatric diseases are provided in the table below.

| Neuro- | |
|---|---|
| psychiatric | |
| Disorder | Animal Model |
| Learning | Presenilin mutant mice (U.S. Pat. No. 6,020,143) |
| Disorders | |
| ADHD | Dopamine depleted rats (see, e.g., Shaywitz et al., 1976, |
| | Nature 261: 153-155; Shaywitz et al., 1976, Science 191: |
| | 305-307, 1976) |
| Eating | Serotonin receptor deficient animals (see, e.g., |
| Disorders | U.S. Pat. No. 5,698,766) |
| Tourette's | Emerich et al., 1991, Pharmacol. Biochem. Behav. 38: |
| Syndrome | 875-880 |
| Dementia | Partial or total loss of function ApoE mutants (see, e.g., |
| | U.S. Pat. No. 6,046,381); mice carrying amyloid precursor |
| | protein genes under the regulation of the platelet-derived |
| | growth factor beta receptor promoter element (Games et al., |
| | 1995, Nature 373: 523-527); mice carrying Amyloid-beta |
| | genes under the control of neurofilament-light gene |
| | promoters (LaFerla et al., 1995, Nat. Genet. 9: 41-47); |
| | transgenic mice expressing tau and tau phosphorylating |
| | proteins (see, e.g., U.S. Pat. No. 5,994,084) |
| Amnesia | Induced by BF lesions in mice (see, e.g., |
| | U.S. Pat. No. 5,494,917); induced by drug treatment (see, |
| | e.g., U.S. Pat. No. 4,816,481; U.S. Pat. No. 4,877,790) |
| Substance | Animal models of cocaine self-administration (Pickens et |
| Abuse | al., 1968, J. Pharm. and Experimental Therapeutics 161: |
| | 122); Animal model for substance abuse-induced |
| | hemorrhagic stroke (U.S. Pat. No. 5,696,125); other |
| | models, Schuster et al., 1974, “The Use of Animal Models |
| | for the Study of Drug Abuse,” In: Research Advances in |
| | Alcohol and Drug Problems, Gibbens, et al. (Eds.), John |
| | Wiley and Sons, New York, Vol. 1, pp. 1-31; Johansen and |
| | Schuster, 1977, “Procedures for the Preclinical Assessment |
| | of Abuse Potential of Psychotropic Drugs in Animals,” In: |
| | Predicting Dependence Liability of Stimulant and |
| | Depressant Drugs, Travis Thompson and Klaus Unna |
| | (Eds.), University Park Press, Baltimore, pp. 203-229; |
| | Weeks, 1962, Science 138: 143-144; Altshuler et al., 1980, |
| | Life Sci. 26: 679-688; Goldberg et al., Science 214: |
| | 573-575 (1981) |
| Schizo- | Amphetamine models, Robinson et al., 1986, Psychol. Bull. |
| phrenia | 88: 551-579; exposure to neurotoxins, |
| | U.S. Pat. No. 5,549,884; transgenic animals with modified |
| | psychosis protecting protein, U.S. Pat. No. 5,962,664; |
| | others, Braff and Geyer, 1990, Arch. Gen. Psychiatry 47: |
| | 181-188 |
| Aggression | rats, U.S. Pat. No. 5,833,945; Albert et al., 1992, |
| | Neuroscience Biobehav. Rev. 15: 177-192; mice, Sadou |
| | et al., 1994, Science 265: 1875-1878; monkeys, Raleigh |
| | et al., 1991, Brain Research 559: 181-190; macaques, |
| | Botchin et al., 1993, Neuropsychopharmacology 9: 93-99; |
| | others, Sheard, 1977. “Animal Models of Aggressive |
| | behavior” In Animal Models in Psychiatry and Neurology, |
| | Pergamon Press Oxford, pp 247-257. |
| Depression | reviewed in Willner, 1991 TiPS 12: 131-136; Willner, |
| | 1990, Pharmac. Ther. 45: 425-455; and Uzunove et al., |
| | 1990, Proc. Natl. Acad. Sci. USA 95: 3239-3244. |
| Anxiety | reviewed in Heilig et al., 1989, Psychopharmacol. 98: 524; |
| | rats, Overstreet, 1993, Neurosci. Biobehav. Rev. 17(1): |
| | 51-68 |
| Obsessive | rat, Szechtman et al., 1999, Pol. J. Pharmacol. 51(1): |
| Compulsive | 55-61; dog, Rapoport et al., 1992, Arch. Gen. Psychiatry. |
| Disorder | 49(7): 517-21; others, Cohen et al., 2000, Eur Neuro- |
| | psychopharmacol. 10(6): 429-35; Adamec, 1999, Physiol. |
| | Behav. 65(4-5):7 23-37, Pare et al., 1996, Biol. |
| | Psychiatry 39(9): 808-13 |
| Sleep | FIV-infected cats, Prospero-Garcia et al., 1994, Proc. Natl. |
| Disorder | Acad. Sci. USA 91(26): 12947-51; rats, Szymusiak et al., |
| | 1993, Brain Res. 629(1): 141-5; Vogel et al., 1990, |
| | Neurosci. Biobehav. Rev. 14(1):77-83; dogs, Faull et al., |
| | 1982, Brain Res. 242(1): 137-43 |

Non-human animals which are genetically engineered to express altered doses of forms of neurally expressed genes are also encompassed within the scope of the invention and include, but are not limited to: transgenic mice, rats, swine, dogs, rabbits, non-human primates (e.g., such as monkeys), and the like. Methods for generating theses animals are known in the art. For example, methods of introducing transgenes into cells are described in U.S. Pat. No. 4,873,191; Palmiter and Brinster, 1986, Ann. Rev. Genet. 20: 465-499. Methods for generating transgenic mice are described in Jaenisch, 1988, Science 240: 1468-1474. Methods for generating transgenic rabbits, sheep, and pigs are described in Hammer et al., 1985, Nature 315: 680-683; Kumar et al., U.S. Pat. No. 5,922,854; and U.S. Pat. No. 6,030,833. Methods of generating transgenic chickens are described in Salter et al., 1987, Virology 157: 236-240), while methods for generating transgenic monkeys are described in Vogel, 2001, Science 291(5502): 226. The entirety of these references are incorporated by reference herein.

Tissues from a non-human animal genetically engineered to over-express or under-express desired genes can be arrayed on microarrays 13. In one embodiment, a microarray 13 is provided comprising tissues from non-human animals expressing different doses of a neurotransmitter pathway gene. Nonlimiting examples of such animals are described in Drago et al., 1994, Proc. Nail. Acad. Sci. USA, 91(26): 12564-12568 (mice lacking D1A dopamine receptors); Calabresi et al., 1997, J. Neurosci. 17(12): 4536-4544 (mice lacking D2 receptors); Silva et al., 1992, Science 257(5067): 206-211 (mice lacking the adenosine A2a receptor); Harris, 1995, Proc. Nail. Acad. Sci. USA 92: 3658-3662 (mice lacking the gamma isoform of protein kinase C); DeVires et al., 1997, J. Neuroendocrinol. 9(5): 363-368; 1997 (oxytocin knockout mice); Konig et al., 1996, Nature 383(6600): 535-538 (mice deficient in pre-proenkephalin); Rosahl et al., 1993, Cell 75(4): 661-70 (mice lacking synapsin I); Lijam et al., 1997, Cell 90(5): 895-905 (mice lacking Dv11), Signorini et al., 1997, Proc. Natl. Acad. Sci. USA 94(3): 923-927 (mice lacking G protein coupled, inwardly rectifying K⁺ channel GIRK2); Aiba et al., 1994, Cell 79(2): 377-388 (mGluR1 deficient mice); Yokoi et al., 1996, Science 273(5275): 645-647 (mGluR2 deficient mice); Masu, 1995, Cell 80: 757-765 (mGluR6 deficient mice); Jang et al., 2000, Brain Res. Mol. Brain Res. 78(1-2): 204-206 (mice lacking the muopioid-receptor gene); and Cremer et al., 1994, Nature 367(6462): 455-559 (NCAM deficient mice). The entirety of these references are incorporated by reference herein.

In some aspects, a microarray 13 comprises samples from a plurality of cultured cells (e.g., from cell lines or primary cell cultures) which have been genetically engineered to express altered doses of neurally expressed genes or modified forms of such genes. In this embodiment, the cells can be either stably or transiently transfected cells.

In still other aspects, the tissue microarray 13 comprises tissues from different recombinant inbred strains of individuals (e.g., such as mice) which differ at only one or a few (less than ten) genetic loci (e.g., comprising different MHC alleles). In a further embodiment, tissues from humans comprising a characterized haplotype are arrayed (e.g., a particular grouping of HLA alleles).

Construction of Microarrays

In one aspect, microarrays 13 are generated by obtaining donor tissues from any of the donor samples described above, embedding these samples, and obtaining portions of the embedded samples for placement in a recipient block or a block of embedding matrix which subsequently can be sectioned, each section being placed on any of the substrates described above. Recipient blocks can be stored indefinitely (e.g., in a refrigerator or freezer unit) for generation of microarrays 13.

Embedding Samples: Forming Donor Blocks

In one aspect of the invention, samples (e.g., cells or tissues) are obtained and either paraffin-embedded, plastic-embedded, or frozen. Methods of fixing tissue samples are described in U.S. Patent Application Serial No. 60/234,493, filed Sep. 22, 2000, the entirety of which is incorporated by reference herein.

Cell samples can be obtained from suspensions of cells (e.g., cells suspended in a bodily fluid, a cell culture medium, or a buffer) and/or can be purified cells (e.g., flow sorted cells or ficoll hypaque collected cells) comprising at least about one cell and preferably at least about 50, at least about 10², 10³, 10⁴, 10⁵, 10⁶, 10⁷, or at least about 10⁸ cells. Cells can be embedded in cell blocks as is known in the art and are preferably fixed prior to embedding as described in U.S. Provisional Application Serial No. 60/234,493, filed Sep. 22, 2000, for example.

In one aspect, cells are deposited in a gel-forming medium, such as an algin medium, and the cell/gel combination can be enclosed in an enclosure such as a support web or plastic block while the gel solidifies. The cells and gel can be co-centrifuged together prior to being enclosed in the enclosure. Cells additionally, or alternatively, can be embedded in paraffin, plastic, or a cryogenic embedding media as is known in the art. The generation of cell blocks is described in EP 408,225, U.S. Pat. No. 4,822,495, U.S. Pat. No. 5,137,710, U.S. Pat. No. 5,817,032, and U.S. Pat. No. 4,656,047, the entireties of which are incorporated by reference herein. After hardening, the cell donor block like the tissue donor block can be further processed as described below.

Forming the Recipient Block

In one embodiment, microarrays 13 according to the invention are constructed by coring holes in a recipient block comprising an embedding substance (e.g., paraffin, plastic, or a cryogenic media) and placing a tissue sample or cell sample core from a donor block in a selected hole. Holes can be of any shape and size, but are preferably made in a regular pattern. In one embodiment of the invention, the hole for receiving the sample is elongated in shape. In another embodiment, the hole is cylindrical in shape.

While the order of the donor samples in the recipient block is not critical, in some embodiments, donor samples are spatially organized. For example, donor samples within a microarray 13 will be ordered into groups which represent characteristics of the patients from which the donor samples are derived. In one embodiment, the groupings are based on multiple patient parameters that can be reproducibly defined from the development of molecular disease profiles. In another embodiment, donor samples are coded by genotype and/or phenotype (e.g., such as according to a particular DSM-IV classification).

In some aspects, samples are obtained which fail to express, or which express altered levels or forms, of a pathway molecule associated with a neuropsychiatric disorder. For example, recipient blocks can be generated by obtaining tissue samples from tissues which fail to express early, middle and late neurotransmitter pathway genes. As used herein, “early pathway genes” are genes whose expression effects the expression of multiple downstream genes (at least about 5), such that perturbing the expression of these genes will effect multiple genes in the pathway. “Middle pathway genes” are genes whose expression is required for the expression of at least about 2 but less than five downstream genes, while “late genes” are those which are downstream in the pathway and whose expression effects only one or a few (e.g., less than about 2 pathway molecules). Recipient blocks comprising tissues/cells having defects in the expression of early, middle and late pathway genes can be generated by obtaining tissue sections of an embedded sample (e.g., a donor block), and subsequently coring the sample if it produces the desired pattern of expression. Recipient blocks are validated by obtaining representative section(s) of the block and reacting the sections with a plurality of molecular probes which can react with early, mid, and late pathway genes and their products (which may include the expression products of other genes or various metabolites or cellular constituents).

Samples on the microarray 13 can also be arranged according to expression of biomolecules, if this is known, or according to characteristics of the source of the sample, including diagnosis (e.g., DSM-IV classification) or prognosis, exposure of the source of the sample to particular treatment approaches, treatment outcome, or according to any other scheme that facilitates the subsequent analysis of the samples and the data associated with them.

The recipient block can be prepared while samples are being obtained from the donor block. However, in one embodiment, the recipient block is prepared prior to obtaining samples from the donor block, for example, by placing a fast-freezing, cryo-embedding matrix in a container and freezing the matrix so as to create a solid, frozen block. The embedding matrix can be frozen using a freezing aerosol such as tetrafluorethane 2.2 or by any other methods known in the art. The holes for holding samples can be produced by punching holes of substantially the same dimensions into the recipient block as those of the donor frozen samples and discarding the extra embedding matrix.

Information regarding the coordinates of the hole into which a sample is placed and the identity of the sample at that hole is recorded, effectively addressing each sublocation 13s on the microarray 13. In one aspect, data relating to one or more of tissue/cell type, morphology, expression of biological characteristics (e.g., expression of gene products), DSM-IV classification and/or other diseases to which the source of the tissue/cell has been exposed, such as concurrent or underlying illnesses, and other information regarding the source of the sample, are recorded and stored in a database, indexed according to the location of the sample on the microarray 13. Data can be recorded at the same time that the microarray 13 is formed, or prior to, or after, formation of the microarray 13.

The coring process can be automated using core needles coupled to a motor or some other source of electrical or mechanical power. Methods for automating tissue arraying are described in U.S. Pat. No. 6,103,518, in International Applications WO 99/44062 and WO 99/44062, in U.S. patent application Ser. No. 09/779,753 entitled “Frozen Tissue Microarrayer,” filed Feb. 8, 2001, and in U.S. patent application Ser. No. 09/779,187 entitled “Stylet For Use With Tissue Microarrayer and Molds,” filed Feb. 8, 2001, the entireties of which are incorporated by reference herein.

In one aspect, the microarrays are “small format microarrays” which comprise donor samples of about 0.6 mm in diameter. Small format microarrays comprise at least about 10, at least about 50, at least about 200, at least about 500, at least about 1000, or at least about 2000 samples arrayed on a single substrate. Large formats microarrays 13 can also be provided comprising at least one sublocation greater in at least one diameter than about 0.6 mm, about 1.2 mm and/or about 3.0 mm. Methods of constructing large format microarrays 13 are disclosed in U.S. patent application Ser. No. 09/780,982, filed Feb. 8, 2001, entitled, “Large Format Microarrays”, the entirety of which is incorporated by reference herein.

In general, large format microarrays comprise at least one sample comprising at least about two different cell types or at least one cell type and an extracellular material (e.g., at least two of proliferating cells, non-proliferating cells, stromal cells, extracellular matrix, myelin, neurofibrillary tangles, necrotic cells, and apoptotic cells). Large format microarrays enable detection of the expression of heterogeneously expressed biological characteristics (e.g., such as gene products) which are expressed in less than about 80% of cells, and preferably in less than about 50%, less than about 20%, less than about 10% or less than about 1% of cells in a sample at a given sublocation 13s on a microarray 13. Generally, fewer than about 50 tissue samples are provided on a single substrate for a large format microarray.

In some applications, such as where a limiting amount of sample is available to be analyzed, an ultrasmall format microarray is generated comprising at least one tissue sample about 0.3 mm or smaller. Microarrays comprising tissue samples of varying sizes can also be provided (i.e., including at least two of any of large format, small format, and ultrasmall format tissue samples). Preferably, different sizes of tissue from the same tissue block are provided. Such microarrays can be used to validate that biomolecules detected in a large format microarray will also be detectable in a small format or ultrasmall format microarray.

Tissue Information System for Evaluating Physiological and Behavioral Responses

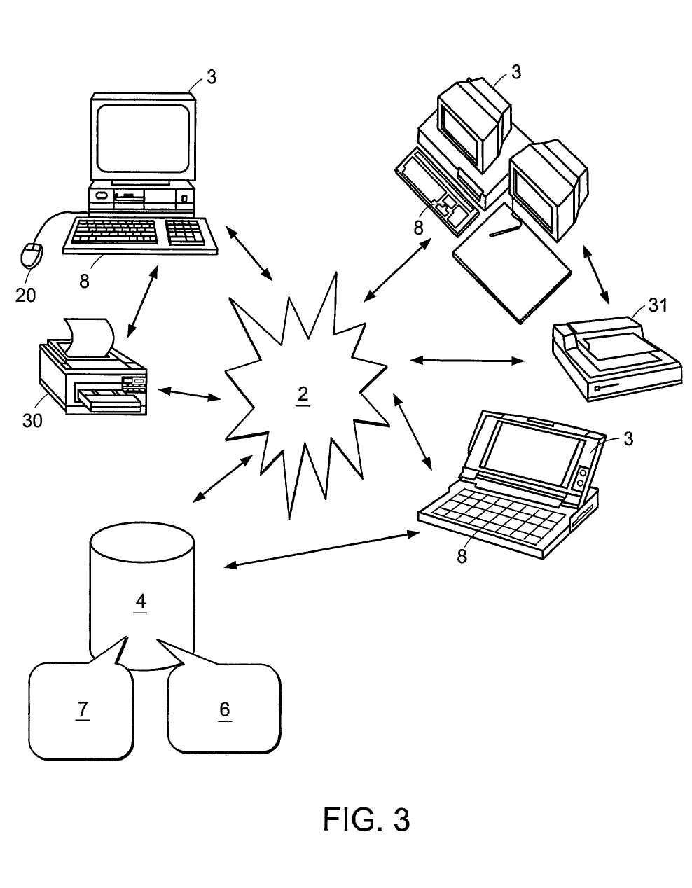

The invention provides a tissue information system 1 (shown in FIG. 3) for evaluating patient responses to neuropsychiatric diseases. The system 1 enables a user to access, organize, and display information stored in a specimen-linked database 5 which includes information relating to samples arrayed on microarrays 13. Data within the specimen-linked database 5 is indexed using identifiers (e.g., such as alphanumeric characters) which identify the tissue microarrays 13 and which are provided to users of the system 1 to enable them to access the database 5. Preferably, the patient responses being evaluated include changes in the expression of a plurality of biological characteristics in response to a neuropsychiatric disorder. More preferably, the responses also include physiological responses and/or behavioral responses to a neuropsychiatric disorder.

The tissue information system 1 comprises at least one user device 3 connected to a network 2. In one embodiment, the network is wide area network (WAN) to which the at least one user device 3 is directly connected. However, in another embodiment, user device 3 is connected to a WAN indirectly through a local area network (e.g., via a proxy server).

Because the user device 3 is connected to the network 2, individual steps of accessing, organizing, and displaying can be performed on one, or a plurality, of user devices 3 at different physical locations. Thus, in one embodiment of the invention, one or more tissue microarrays are each screened at physically distant locations, for example, in different laboratories, hospitals, or companies, and the information obtained from the microarrays screened at each location is correlated with tissue information included within the specimen-linked database 5. Multiple users can both access and add to information within the database 5.

Accessing the system 1 through the user device 3 results in an interface 6 being displayed on a display of the device 3. The interface 6 comprises at least one link to the specimen-linked database 5 which comprises tissue information. In one embodiment, the database 5 is also coupled to an information management system (IMS) 7 which comprises both search functions and relationship determining functions for presenting information to the user in a useable form (e.g., displayed on the device 3).

The device 3 comprises a processor and further includes processor readable storage media or electronic memory that can be accessed by the processor. Processor media includes volatile and nonvolatile media, such as RAM, ROM, EPROM, flash memory, CD-ROM, digital versatile disks (DVD), optical storage media, cassettes, tape, discs, and the like. The device 3 can further include multimedia rendering functions by including audio and video components (not shown). In one aspect, the device 3 also comprises an operating system (e.g., such as Microsoft Windows, UNIX X-Windows, or Apple Macintosh System) and one or more application programs, including an Internet or Web browser, such as Microsoft's Internet Explorer™, or Netscape® (see, as described in Internet Starter Kit by Adam Engst, Corwin Low and Michael Simon, Second Edition, Hayden Books, 1995, the entirety of which is incorporated by reference herein).

Web browsers enable a user of the user device 3 to click on portions of an interface 6 displayed on the display of a user device 3, triggering a response by the system 1. In one aspect, the response by the system 1 is to download and display tissue information on the interface 6 or to provide links to sources of tissue information. In addition to browsers, other networking systems can be included in the tissue information system 1, such as routers, peer devices, common network nodes, modems, and the like.

Suitable devices 3 connectable to the network 2 which are encompassed within the scope of the invention, include, but are not limited to, computers, laptops, microprocessors, workstations, personal digital assistants (e.g., palm pilots), mainframes, wireless devices, and combinations thereof. In one embodiment, the device 3 comprises a text input element 8, such as a key board or touch pad, enabling the user to input information into the system 1. In another aspect, navigating devices 20 are coupled to the device 3 to allow the user to navigate an interface 6. Navigating devices 20 include, but are not limited to, a mouse, light pen, track ball, joystick(s) or other pointing device.

In one aspect, the system 1 comprises at least one server 4. The server 4 provides access to one or more data storage media such as hard disks or hard disk arrays. In one embodiment, the server 4 maintains the database 5 on one of these hard disks. In one embodiment, the server 4 comprises one or more applications, including the IMS 7, which permits a user to access information within the database 5, as well as to implement programs for determining relationships between data in the database 5 and tissues on the microarray 13. In another aspect, another application program is provided which implements the search function of the IMS 7. In a further aspect, application programs which retrieve records also perform user-defined operations on the records (e.g., such as creating folders in which to store records of particular interest to a user). Applications programs ordinarily are written in a general purpose host programming language, such as C<++>; however, such programs can also include user-defined statements written in a relational query language such as SQL. In some embodiments, a web application is provided which includes executable code necessary for the generation of SGL statements. The application can include configuration files which include pointers and addresses to the various software applications included within the server as well as to external and internal databases that must be accessed to service user requests.

In further embodiments of the invention, the system 1 comprises information out put modules 30 (e.g., printers) for outputting and reporting information from the database 5. The system can also comprise information input modules 31 (e.g., scanners), for receiving information from a user, such as scanned data.

In still another embodiment of the invention, a molecular profiling system is provided which is connectable to the device 3. In one embodiment, molecular profiling data is automatically inputted into the database 5, and a user accessing the system 1 has immediate access to this data. Molecular Profiling systems are described in U.S. patent application Ser. No. 09/781,016, “Specimen-Linked Database,” filed Feb. 9, 2001.

Specimen-Linked Database

Information within the specimen-linked database 5 is dynamic, being added to and refined as additional users access the database 5 through the system 1. In one embodiment, inputted information at least comprises information relating to the analyses of the microarrays 13 described above and the database 5 organizes this information according to a data model. Data models are known in the art and include flat file models, indexed file models, network data models, hierarchical data models, and relational data models. Flat file models store data in records composed of fields and are dependent upon the particular applications comprising the IMS 7, e.g., if the flat file design is changed, the applications comprising the IMS 7 must also be modified. Indexed file systems comprise fixed-length records composed of data fields and indexes which group data fields according to categories.

A network data model also comprises fixed-length records composed of data fields which are indexed according to categories. However, network data models provide record identifiers and link fields to connect records together for faster access. Network data models further comprise pointer structures which provides a shorthand means of identifying linked records. Hierarchical data models comprise fixed-length records composed of data fields, indexes, record identifiers, link fields, and pointer structures, but further represent the relationship of different records in a database in a tree structure. Hierarchical data models are described further in U.S. Pat. No. 5,980,096, the entirety of which is incorporated by reference herein.

In contrast, relational data models comprise tables comprising columns and rows of data elements or attributes. Attributes provide information about the different facts stored within the database 5. Columns within the table comprise attributes of the same data type (e.g., in one embodiment, all information relating to patient X's drug exposure), while each row of the table represents a different relationship (e.g., row one, representing dosage, row two representing efficacy, row three representing safety). As with network data models, and hierarchical data models, relational database models link related information within the database. Any of the data models described above can be used to organize information within the database 5 into information categories to facilitate access by a user of the tissue information system 1. In a preferred embodiment, a system operator, i.e., the user who provides access to the tissue information system to other users, determines the parameters which define a particular information category recognized by a particular data model. 110 For example, in one embodiment, the system operator determines the fields that are used to define the information category “drug exposure.” In this embodiment, the system operator may determine that these fields should include: “types of drugs to which the patient was exposed;” “frequency of exposure;” “dose at each exposure;” “physiological response to exposure;” “tests used to measure physiological responses;” “molecular response to exposure;” “tests used to measure molecular responses,” “behavioral responses” and “tests used to measure behavioral responses,” and the like. Similarly, the system operator may determine that fields which define the information category “medical history of a patient” should encompass all information obtained by health care workers at any time during the patient's life, as well as information relating to tests performed by health care workers, or should encompass only selected portions of such records. It should be obvious to those of skill in the art that information categories determined by the system operator can overlap in the types of information contained within them. For example, information relating to medical history could include information relating to a patient's drug exposure. In one embodiment, therefore, the system 1 further comprises links between different information categories which comprise areas of overlap.

The parameters defined by the system user are included within a database dictionary portion of the database 5 and in one embodiment, a user other than the system operator can access the database dictionary, preferably on a read-only basis, to determine what parameters were used to define a particular information category. In another embodiment of the invention, a user of the system can request that additional parameters be included in the definition of an information category, and, subject to the approval of the system operator, the definition of the information category can be modified as the database expands. In a further embodiment, the database 5, for example, as part of the dictionary can include a table comprising word equivalents to facilitate searching by the IMS-7. In some aspects, the table comprises codes representing community accepted definitions of diagnoses, anatomic locations, and the like (e.g., such as SNOWMED codes, DSM-IV-TR codes) or accepted genetic nomenclature (e.g., UNIGENE codes).

In one aspect, new information inputted into the system 1 is stored within a temporary database and is subject to validation by the system operator prior to its inclusion in a portion of the database 5 to which all users of the system 1 have access to.

In another aspect, data within the temporary database, is fully able to be accessed and compared to information within the specimen-linked database 5; however, users of the system 1 are alerted to the fact that data within the temporary database have not necessarily been validated (e.g., repeated or evaluated as to quality). In this embodiment, the information categories included within the temporary database can include information relating to the time and date on which the new information was inputted into the system 1.

In one embodiment of the invention, information within information categories is derived from an analysis of any of the tissue microarrays described above. For example, in one embodiment, the database 5 comprises information reflective of “whole body microarrays” which have been evaluated by user(s) (e.g., microarrays comprising tissue samples from at least about five different tissues, and preferably at least about ten different tissues from a patient). In this embodiment, information included within the database encompasses information relating to the types of tissue on the microarray and relating to biological characteristics of the tissue source (e.g., such as patient information). In another embodiment, the database 5 comprises information including, but not limited to, the sex and age of the tissue source, underlying diseases affecting the tissue source, the types of drugs or other therapeutic agents being taken by the tissue source, the localization of the drugs and agents in the different tissues of the microarray, and the effects of the drugs and agents on the different tissues of the microarray, environmental conditions to which the tissue source has been, and is being exposed to, as well as the lifestyle of the tissue source (e.g., moderate or no exercise, alcohol use, tobacco consumption, and the like), cause of death, and age of death (if appropriate).

In preferred aspects, information relating to microarrays derived from tissues/cells from populations of patients is stored in the database. More preferably, information relating to the biological characteristics of normal patients or patients with the same demographic characteristics as test patients (e.g., having the same underlying or concurrent illnesses) except for the presence of a neuropsychiatric disorder is also included within the database 5.

Preferably, where brain tissue is provided on the microarray, the database 5 includes information relating to the region of the brain and/or types of cells provided at a particular sublocation on the microarray. In one aspect, where cells are obtained from living patients, information in the database 5 can include information relating to neurotransmitter expression in these patients (e.g., such as information obtained from PET scans of patients used to monitor neurotransmitter receptor density in the brain).

In one aspect, this information relates to the expression of genes and/or to the morphological features of samples within the array and the samples represent different stages in the progression of a neuropsychiatric disorder (e.g., for a patient with bipolar disorder, samples from patients in a manic phase and samples from patients in a depressive phase are both provided on microarrays 13, and information relating thereto included in the database). Preferably, patient information, including information relating to the behavioral responses of patients also is included within the database 5.

For example, information relating to responses to questionnaires designed to evaluate a patient's psychotherapy progress also can be included in the database 5. Constant data (e.g., such as patient demographic data, presentation problems, and treatment expectations) can be included in one portion of the database 5 (e.g., a set of records), while variable data (e.g., such as measures of distress and/or well-being) can be stored in other portions of the database 5, thereby providing a “mental health index” for the patient (see, e.g., as described in U.S. Pat. No. 5,435,324, the entirety of which is incorporated by reference herein).

Each of these portions of the database 5 can be cross referenced to each other and to portions of the database comprising molecular profiling data (e.g., gene expression data) obtained from tissue microarrays derived from the patient who answered the questionnaires. Preferably, the microarrays comprise cell samples (e.g., such as blood cell samples) obtained at each time a questionnaire is completed and information relating to the relationship between changes in the mental health status of the patient and changes in the patient's molecular profile is stored within another portion of the database. The mental health index additionally, or alternatively, can be determined from evaluations of the patient by health care workers (e.g., such as psychologists, psychiatrists, social workers, and the like).

While in one embodiment, the database 5 comprises information relating to human tissues, in another embodiment, the database 5 also includes information obtained from non-human patients. For example, in one aspect, the database 5 includes information relating to the biological characteristics of tissues from an animal model of a neuropsychiatric disorder. Preferably, the database 5 also includes information relating to the biological characteristics of tissues from the same animal model but relating to animals which have been exposed to any of drugs, antibodies, protein therapies, gene therapies, antisense therapies, and the like. In some embodiments, the biological characteristics of tissues from non-human patients which have been genetically engineered to over express or under express desired genes (e.g., such as neurotransmitter pathway genes) are included within the database 5. In a preferred embodiment, information relating to the behavioral responses of these non-human patients also is included in the database 5. In a further aspect, information within the database 5 includes information from cultured cells which have been genetically engineered to overexpress or underexpress or ectopically express desired genes. The database 5 can also include information relating to tissues from recombinant inbred strains of individuals (e.g., mice). Such information includes, but is not limited to, information relating to an allele carried at one or more loci in such animals, haplotype information, information relating to the expression of one or more proteins encoded by these loci, and to behavioral responses of these animals to stimuli. In a further embodiment, information relating to diseases associated with particular alleles or haplotypes are further included within the database.

While in one embodiment, information within the database 5 is obtained from tissues/cells provided on the microarrays 13 described above, tissue/cell information can also be obtained from a variety of other sources, such as test samples assayed alongside the microarrays 13 (e.g., using profile array substrates), or from test samples which have been assayed independently of tissue microarrays 13, or from samples from cultured cells, or from tissue panels from living patients or from archived tissues, and the like. Information relating to nucleic acid microarrays, protein, polypeptide, peptide, and other biomolecule arrays can also be included within the database, irrespective of whether information from a corresponding tissue/cell microarray 13 has also been obtained. As used herein, although the database 5 is described as being “specimen-linked” the database can also include data unrelated to specific test specimens.

Information within the specimen-linked database 5 can be organized to facilitate information retrieval by the IMS 7 by providing a plurality of “subdatabases,” each of which comprises information relating to a particular category of tissue/cell information. For example, in one embodiment, the subdatabases comprise information relating to tissues/cells obtained from patients classified as fitting a particular DSM-IV-TR profile (see, e.g., http://www.behavenet.com/capsules/disorders/dsm4classification.htm#). Preferably, a database comprising information from patients classified according to at least 10, at least 20, at least 100, different DSM-IV classifications is included, each DSM-IV classification being used to index a separate portion or “subdatabase” of the database.

In one aspect, subdatabases are restricted to particular types of information and include, but are not limited to, sequence subdatabases, protein structure subdatabases, chemical formula/structure subdatabases, expression pattern or molecular profile subdatabases (e.g., providing information relating to the expression of genes in different tissues), subdatabases comprising information relating to drug targets and drug leads (e.g., including, but not limited to information relating to compound toxicity, side effects, efficacy, metabolism, drug interactions, and the like), as well as literature subdatabases, medical history subdatabases, psychiatric history subdatabases, demographic information subdatabases, treatment subdatabases, and the like. Information contained within one subdatabase can overlap or be repeating in a portion of another subdatabase.

In one embodiment of the invention, data within the database 5 is defined using SNOMED® Clinical Terms™. For example, different clinical concepts (e.g., neuropsychiatric disease, as well as cardiovascular disease, neurodegenerative disease, autoimmune disease, cancer, reproductive disease, and the like) are assigned unique concept identifiers which are represented within a “Concept Table” within the database 5. Concepts can be defined by codes, such that a string of codes can be used to cross reference data from a plurality of databases and subdatabases. In a preferred embodiment, data is also organized in the database 5 using DSM-IV TR codes.

Preferably, the system l's databases 5 are compatible with one or more external databases, e.g., such as external genomics or proteomics databases, and the like. Therefore, in a preferred aspect, the information within the system's database 5 is structured in a format which enables data to be transferred from an external database into the system's database 5 without loss of information content. Suitable formats which can be used include XML-based formats, such as GEML (Genetic Expression Markup Language), BSML (Bioinformatic Sequence Markup Language), CellML (for the storage and exchange of computer-based biological models), AnatML (for information at the organ level), and FieldML (for storing spatial and temporal information about elements in a CellML or AnatML) (see, as described at http://www.esc.auckland.ac.nz/ sites/ physiome/ anatml/pages/; http://www.oasis-open.org/cover/cellML.html; http://www.physiome.org.nz/sites/physiome/anatml/pages/website_generation.html.)

However, it is contemplated that language formats will evolve and that the database 5 will necessarily to evolve to conform to existing language formats. Therefore, in one aspect, the IMS 7 includes a translation function which comprises an application (for example, stored in an intermediary server) for restructuring binary data streams received from an external database into first language format documents (e.g., such as XML language documents) and/or which can restructure first language format documents (such as XML documents) into binary datastreams which can converted into a form compatible with the existing database 5 (i.e., a second language format documents. Application programs which can translate XML documents to binary datastreams and from binary datastreams back to XML formats are described in U.S. Pat. No. 6,209,124, for example, the entirety of which is incorporated herein by reference.

The database 5 also preferably stores image data relating to tissues/cell samples arrayed on a plurality of microarrays 13, e.g., such as microscopy and histological data and in one aspect, the database 5 stores uncompressed raw data files, such as for example, microscopy and histological data obtained from the tissues/cells. The database 5 preferably stores memory intensive files, and the system's network 2 connection enables high speed (T-1, T-3 or higher) transmission of the data to the user. Program applications for image analysis such as Image-Pro® Express for Windows can be used (available from Media Cybernetics, Silver Spring, Md.).

As discussed above, the specimen-linked database 5 according to the invention makes information available concurrently from a number of different sources to enable a user to practice “genomic medicine,” i.e., to develop diagnostic and treatment modalities based not only on the physiological responses of a patient, but also on the biomolecular responses of a patient. As illustrated in the table below, a genomic medicine database according to the invention comprises a plurality of subdatabases, including, but not limited to, a patient information subdatabase, a medical information subdatabase, a pathology information subdatabase, and a genomic information subdatabase. As can be seen from the table, information in one database may overlap (i.e., be repeated) in another database. For example, a pathology subdatabase can included molecular information relating to a particular disease, just as can a genomics database, and may also include additional information, such as information identifying the correlation between a particular marker and a morphological characteristic.

Genomic Medicine Database

| Patient | Medical | Pathology | Genomic |
|---|---|---|---|
| Information | Information | Information | Information |
| Subdatabase | Subdatabase | Subdatabase | Subdatabase |
| Demographics | Diagnosis | Diagnosis | DNA |
| Life style | Other conditions | Histology | Protein |
| Epidemiology | Concurrent Illness | Clinical Data | mRNA |
| Family History | Medications | Molecular Markers | |
| | Psychological | | |
| | Evaluations | | |
| | Outcome Survival | | |

Physiological Response Database

In a preferred embodiment of the invention, the database 5 comprises information relating to the physiological responses of patients to a neuropsychiatric disorder, including responses to treatment for such a disorder (e.g., such as drugs or psychotherapy). Physiological responses include, but are not limited to, cellular metabolism (and preferably, including neural cellular metabolism), energy metabolism, nucleic acid metabolism, signal transduction, progression through the cell cycle, DNA repair, secretion, subcellular localization and processing of cellular constituents (e.g., including RNA splicing, protein modification and cleavage), cell-cell interactions, growth, differentiation, apoptosis, immune responses, neurotransmission, ion transport (preferably, including transport in neural cells), sugar transport, lipid metabolism, and the like. The database 5 also can include information relating to kinetic parameters which govern physiological responses. For example, the database can include information relating to dissociation constants, Michaelis Menton constants, inhibition constants, catalytic constants, circulating half-life of biomolecules, excretion rates, and the like.

In one aspect, physiological responses are evaluated by monitoring the expression of a plurality of biomolecules representing at least one molecular pathway in a tissue sample (“pathway biomolecules”) and using the database 5 to identify correlations between an expression pattern observed and the likelihood that the source of the tissue sample is suffering from a neuropsychiatric disorder. Preferably, physiological responses are evaluated by monitoring the expression of pathway biomolecules in a plurality of tissues, and more preferably, in whole body microarrays representing different populations of patients which share one ore more traits. Still more preferably, pathway molecules being evaluated included neurotransmitter pathway molecules.

Thus, in one aspect, the specimen-linked database 5 includes a plurality of records comprising information relating to pathway biomolecules and the effects of a neuropsychiatric disorder on the expression of these biomolecules. For example, the database 5 can comprise records relating to biomolecules which are expressed or inhibited upon activation of a particular G-protein coupled receptor or “GPCR pathway biomolecules.” Thus, the database can include information relating to any one or more of a serotonin receptor (e.g., 5-hydroxytryptamine 1A, 1B, 1C, 1D, 1F, 2A, 2C, 5A and/or 5B receptors), an adenosine receptor (e.g., an adenosine A1 receptor, an adenosine A2A, A2B, A3, P2U, and/or P2Y receptor), uridine nucleotide receptor, an adrenergic receptor (e.g., α-1A, 1B, 1C, 2A, 2B, 2C, and/or β-1, 2, and/or 3), angiotensin receptor, bombesin receptor (e.g., bombesin Type 3, Type 4), neuromedin B receptor, gastrin-releasing peptide receptor, bradykin receptor, C5A-anaphylatoxin receptor, a cannabinoid receptor (e.g., Type 1, Type 2, Type A), gastrin receptor, dopamine receptor (e.g., dopamine 1A, 1B, D2, D3, D4), endothelin receptor (e.g., endothelin A, endothelin B) formyl-methionyl peptide receptor, gonadotrophin releasing hormone receptor, glycoprotein hormone receptor, histamine receptor (H1 and/or H2), interleukin-8 receptor (e.g., interleukin 8A and 8B), adrenocorticotrophin receptor, melanocortin receptor, melanocyte stimulating hormone receptor, muscannic receptor (e.g., M1, M2, M3, M4, M5 receptors) neurokinin receptors, olfactory receptors, opioid receptors (delta, kappa, mu, and/or X receptors), opsin (blue or red/green sensitive), parathyroid receptor, secretin receptor, vasoactive intestinal peptide receptor, extracellular calcium-sensing receptor, metabotropic glutamate receptor, prostanoid receptor (EP1, EP2, EP3, EP4), thromboxane receptor, somatostatin receptor (Type 1, 2, 3, and/or 4), Burkitts' Lymphoma receptor, EB1I orphan receptor, EDG1 orphan receptor, G10D orphan receptor, GPR3 orphan receptor, GPR6 orphan receptor, GPR10 orphan receptor, LCR1 orphan receptor, mas oncogene, RDC1 orphan receptor SENR orphan receptor, calcitonin receptor, parathyroid hormone receptor, secretin receptor, vasoactive intestinal peptide receptor, extracellular calcium sensing receptor, a glutamate receptor, or mutated or variant forms thereof, and any biomolecules whose expression is turned on or off upon activation of these receptors, and/or their mutant or variant forms.

In one aspect, the database 5 includes information relating to the expression of at least 10, at least about 20, at least about 50, at least about 100 of these biomolecules in a plurality of different tissues (e.g., such as the whole body microarrays described above).

Most preferably, the biomolecules evaluated are part of a neurotransmitter receptor pathway. Thus, in one aspect, the database 5 comprises information relating to the expression of one or more α₁ adrenoreceptor pathway molecules. The α₁-adrenoreceptors respond to epinephrine and norepinephrine by interacting with Gₚ/G_{q} proteins. All subtypes of the receptors are coupled to phospholipase C and activation of the receptors result in the production of IP₃ and DAG. These second messengers activate voltage dependent and independent Ca²⁺ channels and stimulate protein kinase C, phospholipase A₂ and D, arachidonic acid release and cyclic AMP formation (see, e.g., Harrison et al., 1991, TiPS. 12: 62). Preferably, therefore, the database 5 includes information relating to the expression of any of the α_{1A} adrenoreceptor, α_{1B} adrenoreceptor, α_{1C} adrenoreceptor, and α_{1D} adrenoreceptor, and/or information relating to the expression of epinephrine, norepinephrine, G_{p/q} proteins, phospholipase C, IP₃, DAG, ion channel proteins, GTP, and Ca²⁺ in the body of an organism represented by tissues on tissue microarray(s).

Expression information can include information relating to the localization of one or more receptors in the body. Preferably, neural tissues are arrayed on the microarray to enable evaluation of expression of the one or more receptors in the brain, especially in the hippocampus and cortex and in the PNS (e.g., neurons located in vascular and non-vascular smooth muscle) where these receptors are normally expressed.

In another aspect, the database comprises information relating to the expression of one or more α₂-adrenoreceptor pathway molecules. The α₂-adrenoreceptors mediate their functions through a variety of G-proteins including G₁/Gₒ and inhibit cyclic AMP production. The α₂-adrenoreceptor also stimulates Ca²⁺ influx, phospholipase A₂ and Na⁺/H⁺ exchange, and activates K⁺ channels (see, e.g., Bylund et al., 1995, Ann.N.Y.Acad.Sci. 763: 1). Thus, preferably, the database includes information relating to the expression of any one of: the α_{2A} adrenoreceptor, α_{2B} adrenoreceptor, α_{2C} adrenoreceptor, and/or one or more of adenylyl cyclase, epinephrine, norepinephrine, G_{1/o} proteins, cAMP, voltage-gated Ca²⁺ channel proteins, Ca²⁺-dependent K⁺ channel proteins, GTP, and Ca²⁺ in the body of an organism represented by tissues on a tissue microarray. Preferably, expression information includes information relating to the localization of one or more α₂ adrenoreceptors in the body (e.g., such as in neurons of the CNS and PNS where these receptors are normally expressed).

In still another aspect, the database 5 comprises information relating to β-adrenoreceptor pathway molecules. The β-adrenoreceptors are also coupled via G-proteins to intracellular second messenger systems (Stadet, 1991, In: Molecular Biology, Biochemistry and Pharmacology, Ed. R. R. Ruffolo p 67). The β₁-adrenoreceptor is positively coupled to adenylate cyclase via activation of Gₛ G-proteins as are the β₂- and β₃-adrenoreceptors. However, activation of the β₂- and β₃-adrenoreceptors results in stimulation, or stimulation and inhibition of adenylate cyclase, respectively, while activation of the β₄-adrenoreceptor results in increased cAMP and stimulation of cAMP-dependent protein kinase. β-adrenoreceptors may also be linked to voltage-gated Ca²⁺ channels by stimulatory G-proteins (see, e.g., Bylund et al., 1994, Pharmacol.Rev. 46: 121). Therefore, preferably, the database includes information relating to the expression of: one or more of the β₁ adrenoreceptor, β₂ adrenoreceptor, β₃ adrenoreceptor, β₄ adrenoreceptor and/or one or more of epinephrine, norepinephrine, adenyl cyclase, β-adrenoreceptor kinase, Gs proteins, GTP, and Ca²⁺. Expression information can also include information relating to the localization of one or more β adrenoreceptor receptors in the body. Preferably, expression of the β₁ adrenoreceptor receptor is evaluated at least in the striatum and in cardiac and adipose tissue, while the expression of the β₂ adrenoreceptor receptor is evaluated at least in vascular, uterine, and airway smooth muscle. The expression of the β₃ and β₄ adrenoreceptors are preferably evaluated in at least in adipose tissue and cardiac tissue, respectively, as these are all tissues in which the receptors are normally expressed.

In still another aspect, the database 5 comprises information relating to the expression of dopamine receptor pathway molecules. D1-like receptors (D1 and D5) stimulate adenylyl cyclase and phospholipase C by coupling to Gₛ proteins. D2-like receptors (D2, D3, and D4) inhibit adenylyl cyclase and Ca²⁺ channels, activate K⁺ channels, stimulate arachidonic acid release and MAP kinase pathway molecules (e.g., JIP-1, MLK, HPK, JNK, MEKK1, MKK4, MAPK, cJun, and p38 proteins; see, as described in Chang et al., 2001, Nature 410: 37-40, the entirety of which is incorporated by reference herein). Therefore, in one aspect, the database 5 includes information relating to the expression of one or more of D1, D2, D3, D4, and D5 and/or one or more of dopamine pathway molecules including, but not limited to: PAH enzyme, tetrahydrobiopterin, tyrosine and tryptophan hydroxylases, AP-2, dopamine, L-dopa, dopa decarboxylase (DDC), dopamine-beta-hydroxylase (DBH), catechol-o-methyl transferase, monoamine oxidase, adenylyl cyclase, phospholipase C, Gₛ proteins, cAMP, GTP, and Ca²⁺. In one aspect, the database also includes information relating to the adenosylation of D4 and the expression of methionine adenosyl-transferase (MAT).

Preferably, expression information also includes information relating to the localization of one or more dopamine receptors in the body. For example, the presence of D1 in the caudate/putamen, nucleus accumbens, olfactory tubercle, hypothalamus, thalamus, and front cortex of the brain, the presence of D2 in the caudate/putamen, nucleus accumbens, olfactory tubercle, and cerebral cortex, the presence of D3 in the nucleus accumbens, olfactory tubercule, islands of Calleja, and cerebral cortex, the presence of D4 in the retina, frontal cortex, midbrain, amygdala, hippocampus, hypothalamus, and medulla, and the presence of D5 in the hippocampus, thalamus, lateral mamillary nucleus, striatum, and cerebral cortex, can be evaluated in tissue microarrays 13 comprising neural samples from patients having neuropsychiatric disorders.

The database 5 also can comprise information relating to opioid receptor pathway molecules. Opioid receptors μ, δ, and κ, are coupled to second messengers through pertussis toxin-sensitive G proteins (G₁/Gₒ) and bind to opioid peptides β-endorphin, met- and leu-enkephalins, metorphamides, dynorphins, nociceptin, and endomorphins 1 and 2. Opioid receptor-evoked cellular responses include activation of an inwardly rectifying potassium channel, activation of voltage operated calcium channels, inhibition of adenylate cyclase, activation of phospholipase A₂ (PLA₂), PLC b, activation of MAP Kinase, activation of large conductance calcium channels, inhibition of L and T type voltage operated calcium channels, and changes in gene expression of adenylyl cyclase and activation of the cAMP response element binding protein (CREB) (see, e.g., as described at www.tocris.com/opioidreview.htm). Therefore, in a preferred embodiment, information relating to the expression of one or more of: opioid receptors μ, δ, and κ, Gᵢ proteins, Gₒ proteins, opioid peptides (e.g., β-endorphin, met- and leu-enkephalins, metorphamides, dynorphins, nociceptin, and endomorphins 1 and 2), inwardly rectifying potassium channel proteins, voltage operated calcium channel proteins, adenylate cyclase, phospholipase A₂ (PLA₂), PLC b, MAP Kinase pathway proteins, large conductance calcium channel proteins, L and T type voltage operated calcium channel proteins, CREB, GTP, and Ca²⁺ is monitored, preferably, in neural tissues (e.g., spinal cord tissues and brain tissues) from patients with neuropsychiatric disorders and is stored in the database 5.

The database 5 also can include information relating to the expression of cannabinoid pathway molecules. The CB₁ receptor is a GPCR which inhibits adenylate cylase activity and is responsive to psychoactive cannabinoids. Responses to CB₁ binding include activation of inwardly rectifying K+ channels and MAP Kinases. Thus, in one aspect, information relating to the expression of one or more of a CB, receptor, anandamide (the endogenous receptor ligand), anandamide hydrolase, adenylate cyclase, inwardly rectifying K+ channel proteins, MAP Kinase pathway proteins, GTP, and Ca²⁺, is obtained and is entered into the database 5. Preferably, expression is evaluated at least in tissues in which receptor expression is found (e.g., the hippocampus, basal ganglia, globus pallidus, entopeduncular nucleus, substantia nigra pars reticula, amygdala, hypothalamus, cerebellum, brainstem, spinal testes, sperm, HUVEC cells, and vascular cells, and smooth muscle cells). In another aspect, the database 5 includes information relating to the expression of CB₂ receptor pathway molecules (e.g., such as CB₂, pertussis toxin-sensitive G-proteins, anandamide, anandamide hydrolase, CB₂ receptor, GTP, and Ca²⁺). Tissues evaluated for CB₂ expression can include granulocytes, macrophages, monocytes, spleen tonsils, bone marrow, thymus, pancreas, B cells, natural killer cells, and the cerebellum.

In a further aspect, the database 5 includes information relating to the expression of one or more muscarinic receptor pathway molecules. For example, the database can include information relating to the expression of one or more of the M₁ receptor, M₂ receptor, M₂ receptor, M₃ receptor, M₄ receptor, and M₅ receptor and/or one or more of acetycholine, phospholipase C, Gq/₁₁ proteins, IP₃, NO synthase, GTP, and Ca²⁺. Preferably, information is obtained relating to the expression of the receptors in the brain (to evaluate the expression of M₁, M₄, and M₅), sympathetic postganglion neurons (i.e., to evaluate the expression of M₁), myocardium, smooth muscles, presynaptic sites (to evaluate the expression of M₂), glandular tissue, and in vascular smooth muscle(to evaluate the expression of M₃) of patients with neuropsychiatric disorders and is stored in the database 5.

In one aspect, the database 5 comprises information relating to the expression of one or more AMPA receptor (e.g., GluR1, GluR2, GluR3, and GluR4) pathway molecules. AMPA receptors are ionotropic receptors which mediate fast synaptic transmission and depolarisation. Thus, in one aspect the database 5 comprises information relating to the expression of one or more of GluR1, GluR2, GluR3, GluR4, L-glutamate, L-glutamine, NAALADase, and N-acetyl-L-aspartate-L-glutamate (NAAG)). In another aspect, the database 5 comprises information relating to the expression of one or more Kainate receptors (e.g., GluR5, GluR6, GluR7, KA1, KA2, L-glutamate, L-glutamine, NAALADase, and NAAG). In a further aspect, the database comprises information relating to the expression of one or more NMDA receptors (e.g., NMDA1, NMDA2A, NMDA2B, NMDA2C, NMDA2D, NMDA3A) and/or L-glutamate, L-glutamine, NAALADase, NAAG, glycine, Zn²⁺. Preferably, expression data relating to all of these pathway molecules is monitored at least in neural tissue.

The database further can include information relating to the expression of metabotrobic glutamate (mGlu) receptor pathway molecules. Preferably, this portion of the database 5 is subdivided into subdatabases comprising information relating to Group 1 mGlu receptor (mGlu1 and mGlu5) pathway molecules, Group II mGlu receptor (mGLu 2 and mGlu 3) pathway molecules, and Group III mGlu receptor (mGlu 4, 6, 7, and 8) pathway molecules. Group I receptor are coupled to PLC and intracellular calcium signaling molecules while Group II and III receptors are negatively coupled to adenylyl cyclase.

Therefore, in one aspect, the database comprises information relating to the expression of Group I mGluR1 receptors and one or more of L-glutamate, L-glutamine, NAALADase, N-acetyl-L-aspartate-L-glutamate (NAAG), phospholipase C, G_{q/11} proteins, IP₃, DAG, and Ca²⁺, preferably in neural tissues from patients; and/or information relating to Group II receptor pathway molecules, including one or more of: the mGluR2 receptor, mGluR3 receptor, L-glutamate, L-glutamine, NAALADase, and N-acetyl-L-aspartate-L-glutamate (NAAG), adenylyl cyclase, G₁ proteins, Gₒ proteins, and Ca²⁺ (preferably in CNS tissues from patients). The database also can include information relating to Group III receptor pathway molecules, including one or more of: mGluR3, L-glutamate, L-glutamine, NAALADase, N-acetyl-L-aspartate-L-glutamate (NAAG), adenylyl cyclase, G₁ proteins, Gₒ proteins, GTP, and Ca²⁺.

In yet another aspect, the database 5 includes information relating to the expression of one or more serotonin receptor pathway molecules, i.e., information relating to the expression of one or more of: the 5-HT_{1A} receptor, 5-HT_{1B} receptor, 5-HT_{1C} receptor, 5-HT_{1D} receptor, 5-HT_{1E} receptor, 5-HT_{1F} receptor, and/or serotonin (5-hydroxytryptamine), PAH enzyme, TPH, VMAT2, HTT, and MAO-A proteins, adenylyl cyclase, G₁/Gₒ proteins, GTP, and Ca²⁺; information relating to one or more of 5-HT_{2A} receptor, 5-HT_{2B} receptor, 5-HT_{2C} receptor, and/or PAH enzyme, TPH, VMAT2, HTT, and MAO-A proteins, 5-HTTLPR, serotonin, G_{q} GTP binding protein, GTP, and Ca²⁺; and/or information relating to one or more of: the 5-HT₃ receptor, 5-HT₄ receptor, 5-HT₅ receptor, 5-HT₆ receptor, 5-HT₇ receptor, and/or the PAH enzyme, TPH, VMAT2, HTT, and MAO-A proteins, serotonin transporter gene (5-HTTLPR), serotonin, adenylyl cylcase, Gₛ GTP binding protein, GTP, and Ca²⁺. The database 5 can also include information relating to the expression of one or more neurotrophin family proteins (e.g., BDNF, neurotrophin-3 (NT-3) and neurotrophin-4 (NT-4)) which mediate the turnover of serotonin, and/or one or more serotonin precursor molecules such as 5-HIAA, L-Trp and 5-hydroxytryptophan.

Other pathway molecules whose expression can be evaluated and stored in the database include nicotinic receptor pathway molecules (e.g., one or more of the neuronal, α-bungarotoxin sensitive receptor, the ganglion receptor, the muscle receptor, acetylcholine, dimethylaminoethanol, monoaminoethanol, choline, serine, choline acetylase, and acetylcholinesterase); GABA_{A} receptor pathway molecules (e.g., one or more of GABA_{A} receptor, glutamic acid decarboxylase (GAD), GABA transferase, GABA, L-glutamine, L-glutamate, Cl⁻), GABA_{B} receptor pathway molecules (e.g., one or more of the GABA_{B} receptor, gamma-amino butyric acid, GABA transferase, GABA, L-glutamine, L-glutamate, cAMP, Gₛ proteins, G₁ proteins, K⁺, GTP, and Ca²⁺); and GABA_{C} receptor pathway molecules (e.g., such as one or more of the GABA_{C} receptor, glutamic acid decarboxylase (GAD), GABA transferase, GABA, L-glutamine, L-glutamate, and Cl⁻).

For each of the above neurotransmitter pathways, information relating to expression can be correlated with genotyping information, preferably obtained from the same patients whose tissues/cells are arrayed on the microarrays. For example, in one aspect, a relational subdatabase correlating expression information of one or more pathway molecules with information regarding nucleic acid and/or amino acid polymorphisms in the one or more pathway molecules is provided. In a further aspect, additional subdatabases are provided which include information relating to agonists and antagonists of neurotransmitter receptors, as well as information relating to the expression of the pathway molecules in the presence or absence of the agonists and antagonists. Agonists and antagonists of specific neurotransmitter receptor molecules are described in Watling, K. J., Ed., 1998, In The RBI Handbook of Receptor Classification and Signal Transduction, Sigma Aldrich Biochemicals Incorporated, pp. 10-15, the entirety of which is incorporated herein. The database 5 further preferably includes information relating to the expression of neurotransmitter transporter proteins (see, e.g., U.S. Pat. No. 5,580,775). The entirety of these references are incorporated by reference herein).

Preferably, the database 5 comprises information relating to the expression of modified forms of the various neurotransmitter pathway molecules described above (e.g., such as the receptors) to distinguish between the expression of active and inactive forms of these molecules. Such information can be obtained by performing immunohistochemistry on tissues/cells using antibodies which react specifically with the modified forms and not with the unmodified forms in conjunction with antibodies which specifically recognize the unmodified forms and antibodies which recognize both modified and unmodified forms. Methods of generating such antibodies are known in the art and are described further below.

It should be obvious to those of skill in the art that the pathway molecules described above are non-limiting examples of molecules which interact in various neurotransmitter pathways, and that other molecules exist and are encompassed within the scope of the invention. The expression of multiple neurotransmitter pathway molecules in a single patient can be evaluated using microarrays 13 according to the invention and information relating to this expression stored in the database 5.

Further, in addition to information relating to the neurotransmitter pathway biomolecules exemplified above, information relating to the expression of other neurally expressed molecules can also be included in the database. For example, in one embodiment, information relating to the expression of glial fibrillary acidic protein (GFAP), dihydropyrimidinase-related protein 2, ubiquinone cytochrome c reductase core protein 1, carbonic anhydrase 1 and fructose biphosphate aldolase C in patients can be stored in the database, as all of these have been shown to increase in neural tissues from patients with schizophrenia (see, Johnston-Wilson et al., supra). Additional molecular profiling data can be obtained regarding the expression of such proteins as synapsin Ia, Ib and IIb proteins, D8117 B lymphocyte alloantigen, corticotropin-releasing factor (CRF), the receptor for CRF, adrenocorticotropic hormone (“ACTH”), and other stress related hormones, beta -endorphin, and other pro-opiomelanocortin (“POMC”)-derived peptides, apoE, presenillin, neuronal nitric oxide synthase gene (nNOS1a), Apolipo protein-D (APO-D), uncoupling proteins UCP1 and UCP2, and the like. Further, in some aspects it is contemplated that expression data relating to uncharacterized gene products expressed at least in neural tissue will be stored in the database 5 (e.g., such as EST expression data).

In some aspects, the information relating to the expression of pathway biomolecules expressed in tissue/cell microarrays from patients will be complemented by information obtained from other types of arrays, e.g., such as nucleic acid arrays (e.g., cDNA arrays, oligo arrays, gene chips), protein/polypeptide/peptide arrays and/or other small molecule arrays. Preferably, these arrays are obtained from the same patients who provided the tissue/cell microarrays. In still other aspects, information relating to the expression of biomolecules which are not readily assayable on tissue/cell microarrays may be obtained from patient samples evaluated in non-array based assays. For example, in one aspect, the levels of neurotransmitter metabolites are evaluated in CSF fluid from patients using assays routine in the art (e.g., such as reversed-phase high-performance liquid chromatography as described for example, in Earley et al., 2001, Mov. Disord. 16(1): 144-9, the entirety of which is incorporated by reference herein). In still other aspects, neurophysiological responses being evaluated include electrophysiological data which is preferably being obtained from patients supplying tissues for microarrays. Information relating to such responses is also included within the database 5.

The physiological response database 5 can also include information relating to the effect of drugs on a plurality of pathway molecules and/or information relating to the localization of one or more drugs in tissues on a whole body microarray from one or more patients. Subdatabases including this information can be organized according to particular classes of drugs and particular concurrent and underlying illnesses to which a patient has been exposed or according to other common patient characteristics.

Preferably, the physiological response database 5 comprises information relating not only to the expression of biomolecules in particular pathways, but also includes information relating to the biological impact of this expression. Still more preferably, the database includes information relating the expression of neurotransmitter pathway biomolecules to physiological parameters such as blood pressure, heart rate, pH, body temperature, level of metabolites (e.g., in CSF fluid or blood) and the like. In some embodiments, information relating to biological impact includes the association of the expression of pathway biomolecules with parameters considered as being important to quality of life, e.g., levels of pain, ability to move, sleep, eat, feelings of well being, and the like.

In all the aspects discussed above, control subdatabase(s) also are preferably provided which comprise information relating to the average physiological responses of demographically matched patients who have similar traits as test patients except for the presence of a neuropsychiatric disorder (e.g., such patients can also have one or more non-neuropsychiatric disorders or be without any pathological conditions). Both control subdatabases and test subdatabases (comprising information from patients with neuropsychiatric disorders) can further include information relating to the expression of housekeeping genes in different tissues in patients from different demographic groups to provide a way of normalizing data in the different portions of the database 5.

In a further preferred aspect, the database 5 includes molecular profiling information relating to relatives of patients with neuropsychiatric disorders. For example, in one aspect, sib pair information is obtained (e.g., information from a patient and their brother(s) and/or sister(s)). Information from monozygotic twins is highly desirable. In a further embodiment, information from an at least two generation pedigree is obtained, and preferably, information from an at least three generation pedigree is obtained. In still another aspect, information from an inbred population is provided to the database 5. Preferably, in all of these aspects, this information is linked to tissue/cell samples provided on a plurality of microarrays 13 which are being evaluated to obtain molecular profiling data, and the information is correlated with patient information as described above.

Behavioral Response Database

In one aspect, the portion of the database 5 comprises information relating to behavioral responses associated with a neuropsychiatric disease. Information relating to such responses can be obtained from questionnaires provided to patients. Responses to such questionnaires can be given value scores (see, e.g., as described in U.S. Pat. No. 5,435,324 and U.S. Pat. No. 5,961,332, the entirety of which is incorporated herein by reference) and these scores stored in a relational database with further information about the patient (e.g., such as DSM IV classification, molecular profiling data, and the like).

Data from psychological tests such as the Minnesota Multiphasic Personality Inventory (MMPI) test, the California Psychological Inventor (CPI), and the Sixteen Personality Factor Questionnaire (16PF) can be included within the database 5. Questionnaires can include questions designed to illicit information relating to social problems, threats to well-being of self or others, dissatisfaction with one's job, education, or standard of living (e.g., current external stimuli). Questionnaires can also include intelligence tests, personality tests (e.g., Meyers Briggs tests, and the like) and questions relating to past events (e.g., questions relating to childhood, relationships, abuse or neglect, peer relationships, and the like). In one aspect, the database 5 comprises information obtained from patient session records obtained during psychotherapy and/or before, during, and/or after treatment with medication. In another aspect, the database is a relational database which correlates behavioral response information with time after initial presentation to a health care worker, to record the progress of therapy.

Data can be stored in the database 5 in the form of a matrix or a spreadsheet, for example, organized according to the DSM-IV classification of the patient and/or by other traits (e.g., age, sex, presence of non-neuropsychiatric diseases, drug treatments, and the like). Data groupings can be validated or modified after relationships between data are determined using the IMS 7 as described further below.

A portion of the database 5 can also comprise information relating to treatment options, including, but not limited to, drugs available to patients who exhibit particular behavioral and/or physiological responses. Treatment databases can further include expert rules for correlating particular treatment options to particular responses. Treatment databases are known in the art and are described in U.S. Pat. No. 6,188,988, for example, the entirety of which is incorporated by reference herein.

Information Management System for Identifying Pathway Biomolecules and for Modeling Molecular Pathways

The database 5 according to the invention is coupled to an Information Management System (IMS) 7. In one aspect, the IMS 7 includes functions for searching and determining relationships between data structures in the database 5. In another aspect, the IMS 7 displays information obtained in this process on an interface 6 of the user device 3. IMS 7 programs can be stored within one or more servers 4, and can be accessible remotely by the user of the device 3 through the network 2. In one aspect, the IMS 7 is accessible through a readable medium, which the user accesses through their particular user device 3, e.g., such as a CD-ROM.

IMS 7's encompassed within the scope of the present invention include the Spotfire™ program, which is described in U.S. Pat. No. 6,014,661, the entirety of which is incorporated by reference herein. This database management software provides links to genomics data sources and those of key content and instrumentation providers, as well as providing computer program products for gene expression analysis. The software also provides the ability to communicate results and records electronically. Other programs can also be used, and are encompassed within the scope of the invention, and include, but are not limited to Microsoft Access, ORACLE and ILLUSTRA.

In one asepct, the IMS 7 comprises a stored procedure or programming logic. Stored procedures can be user-defined, for example, to implement particular search queries or organizing parameters. Examples of stored procedures and methods of implementing these are described in U.S. Pat. No. 6,112,199, the entirety of which is incorporated herein by reference.

In another aspect, the IMS 7 includes a search function which provides a Natural Language Query (NLQ) function. In this embodiment, the NLQ accepts a search sentence or phrase in common everyday from a user (e.g., natural language inputted into an interface of a device 3) and parses the input sentence or phrase in an attempt to extract meaning from it. For example, a natural language search phrase used with the specimen-linked database 5, could be “provide medical history of patient providing sample at sublocation 1,1 of microarray 4591.”This sentence would processed by the search function of the IMS 7 to determine the information required by the user which is then retrieved from the specimen-linked database 5. In another embodiment of the invention, the search function of the IMS 7 recognizes Boolean operators and truncation symbols approximating values that the user is searching for.

In one embodiment, the search function of the IMS 7 generates search data from terms inputted into a field displayed on an interface 6 of a device 3 in the system 1 in a form recognized by at least one search engine (e.g., identifying search terms which are stored in fields in the database 5 or in a summary subdatabase), and transfers the search data to at least one search engine to initiate a search. However, in another embodiment, the search query is communicated through the selection of options displayed on the interface 6. For example, in one embodiment, search results are displayed on the interface 6 and may be in the form of a list of information sources retrieved by the at least one search engine. In another embodiment, the list comprises links which link the user to information provided by the information source. In a further embodiment, the search function of the IMS 7 removes redundancies from the list and/or ranks the information sources according to the degree of match between the information source and the search terms extracted, and the interface 6 displays the information sources in order of their rankings. Search systems which can be used are described in U.S. Pat. No. 6,078,914, the entirety of which is incorporated by reference herein.

In another aspect, the search function of the IMS 7 searches a summary subdatabase of the database 5 to identify particular subdatabase(s) most relevant to the search terms which have been inputted by the user. In this embodiment, the search function of the IMS 7 restricts its search to subdatabases so-identified. In a further embodiment, the subdatabases searched by the IMS 7 can be defined by the user.

In one aspect, relationships between records stored in the database 5 are defined by codes, such as SNOMED® codes, which can be inputted into the system by a user (e.g., on an interface of a user device 3). SNOMED® codes are described further in Altman et al., 1994, Proceedings of American Medical Informatics Association Eighteenth Annual Symposium on Computer Applications in Medical Car, November 5-9, Washington D.C. pg. 179-183; Bale, 1991, Pathology 23(3): 263-267; Ball et al., 1999, Computing pp. 40-46; Barrows et al., 1994, Proceedings of American Medical Informatics Association Eighteenth Annual Symposium on Computer Applications in Medical Care, November 5-9, Washington D.C. pg. 211; Beckett, Pathologist, Vol. XXXI, No. 7, July 1977; Bell, 1994, Journal of the American Medical Informatics Association, 1(3): 207-217; Benoit et al., 1992, Proceedings of the Annual Symposium of Computers Applications in Medical Care, pp. 787-788; Berman et al., 1994, Proceedings of American Medical Informatics Association Eighteenth Annual Symposium on Computer Applications in Medical Care, pg. 188-192; Berman et al., 1996, Modern Pathology 9(9): 944-950; Bidgood, 1998, Meth. Inf. Med. 37: 404-414; Brigl et al., 1995, International J. of Bio-Med. Comput., 38: 101-108; Brigl et al., 1994, Int J. Biomed. Comput. 37(3): 237-247; Campbell et al., 1998, Methods Inf. Med. 37 (4-5): 426-39; and Campbell et al., 1994, Proceedings of American Medical Informatics Association Eighteenth Annual Symposium on Computer Applications in Medical Care, Washington, D.C. pg. 201-205, for example, the entireties of which are incorporated by reference herein.

Thus, in a further embodiment of the invention, the IMS 7 includes a mapping function for mapping terms to particular tables/records within the database 5. Alternatively, or in addition to SNOMED®, other classification and mapping codes can be used (e.g., CPT, OPCS-4, ICD-9, and ICD-10). In one aspect, the IMS 7 comprises a program enabling it to read inputted codes and to access and display appropriate information from an appropriate relationship table in the database 5. For example, unique SNOMED® codes can be assigned to tissues from specific anatomic sites (e.g., neural tissues) or can be assigned to tissues having specific pathologies (e.g., such as neurodegeneration or ischemia). In a further embodiment (not shown), information relating to tissue samples/specimens are cross-referenced using SNOMED® codes for both anatomic sites and diagnosis. Exposure of individual tissue samples to particular drugs can also be indicated by codes such as by using American Hospital Formulary Service List (AHFS) Numbers or “V-Codes” to classify other types of circumstances or events to which the source of a tissue sample has been exposed, for example, such as vaccinations, potential health hazards related to personal and family history (e.g., a history of high blood pressure, diabetes, or stroke), exposure to toxic chemicals, and the like (see, e.g., as described in U.S. Pat. No. 6,113,540).

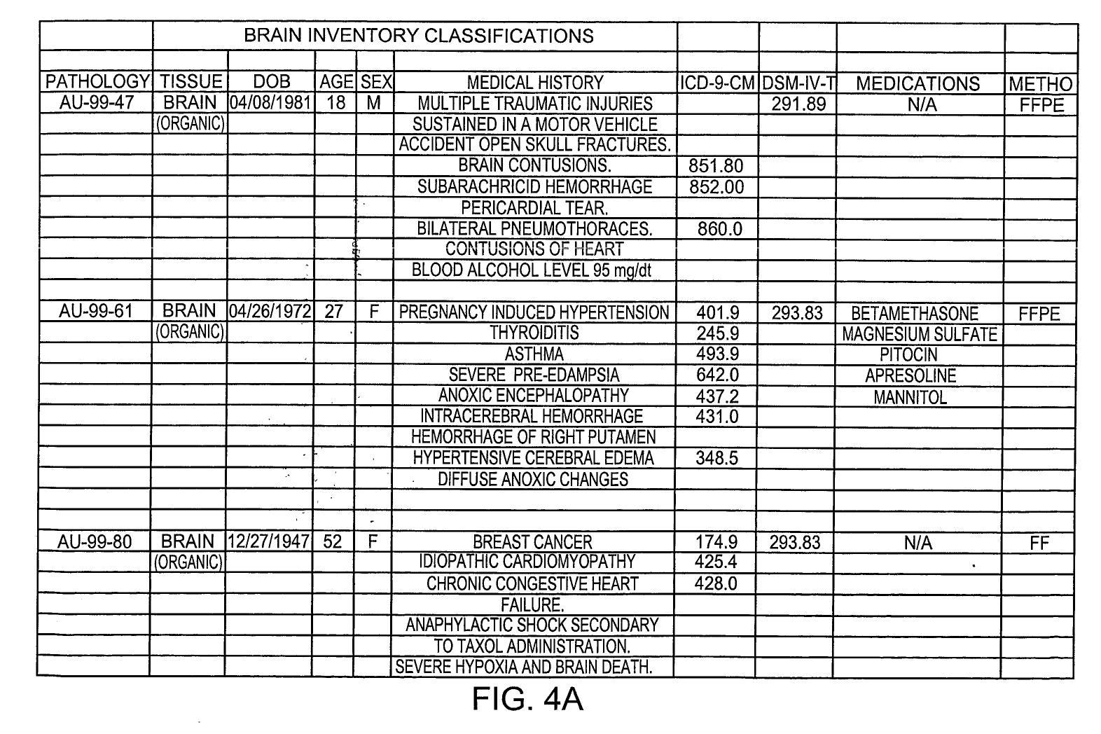

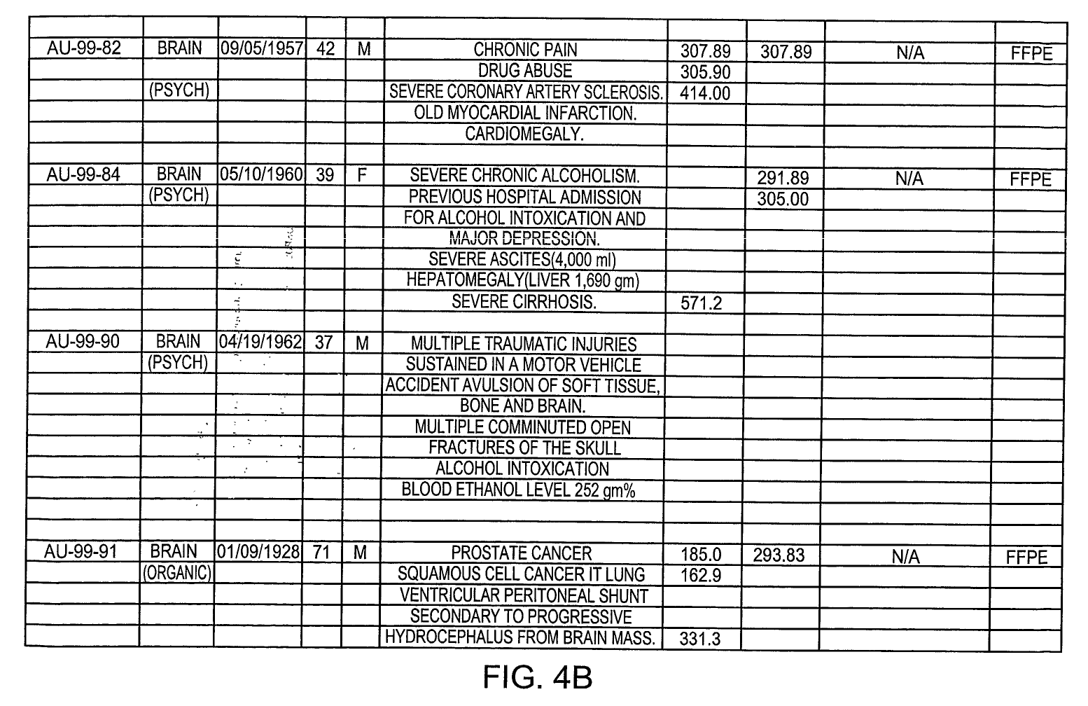

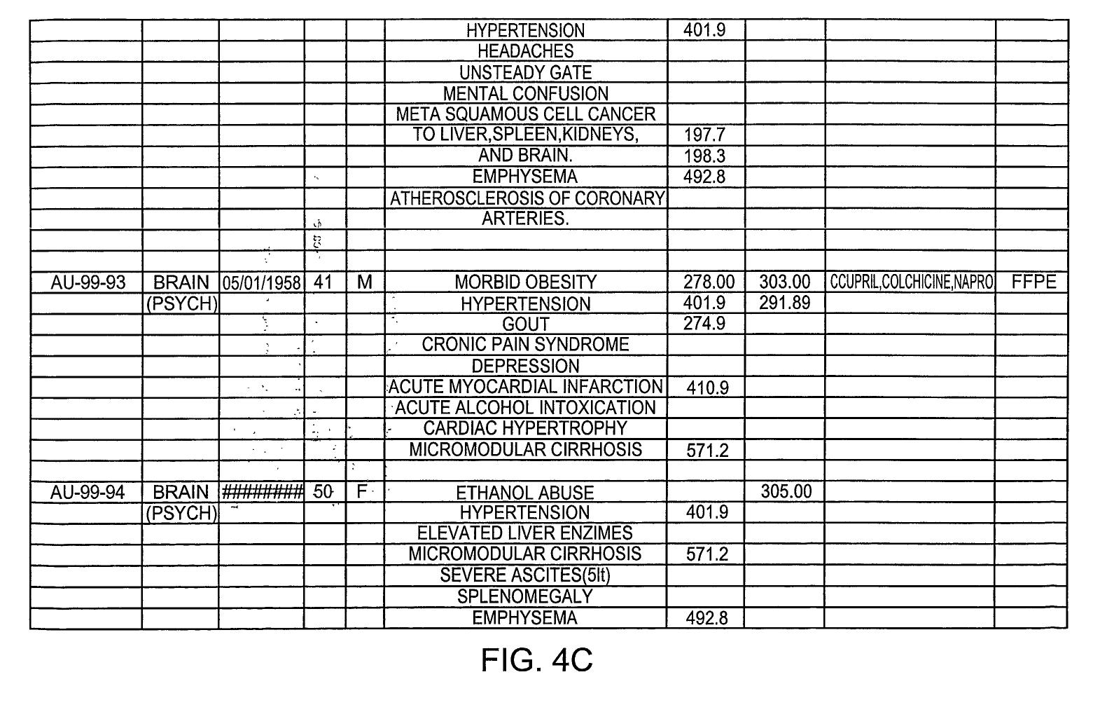

In a preferred embodiment, specimens/tissues on a microarray from patients having a neuropsychiatric disorder are cross-referenced in the database 5 (i.e., linked to the database) according to the patient's classification using DSM-IV-TR criteria. In another embodiment, specimens/tissues are linked to the database using ICD-9-CM criteria. In still another embodiment, as shown in FIG. 4, the specimens/tissues are cross-referenced using a number of criteria, such as tissue type, date of birth of the patient, medical history of the patient, family history, ICD-9 classification, DSM-IV TR classification, medications which the patient is taking, and the like. In a further embodiment, ICD-9 and/or DSM-UV-TR classifications are indicated using codes. ICD-9 and DSM-IV TR codes are described at http:// www.nzhis.govt.nz/projects/dsmiv-code-table.html, for example.

In one aspect, codes or scores are assigned to psychological/behavioral information. As discussed above psychological/behavioral information can be scaled, e.g., such as using though-frequency scores, or emotion-intensity scores. Scores can be assigned by the system user and/or assigned by the IMS 7 according to the relationship between certain kinds of behavior/psychological responses and/or physiological responses and/or molecular profiling data. Values can be multiplied by statistically determined weighting values according to the influence such responses may have on the feelings of well-being or distress of the patient, using known statistical methods. Weighting values can be selected by the user, the system operator, or the IMS 7 (e.g., a high value can be assigned to a response that has a statistically significant association with a particular DSM-IV classification). In a preferred embodiment, the IMS 7 compares such scores to scores of individuals with similar traits (e.g., age, sex, underlying or concurrent illnesses) but who do not have a neuropsychiatric disorder.

Information relating to behavioral profiles can be identified using numerical or alphanumerical identifiers to the confidentiality of this data. Preferably, a user inputting information into the system accesses a portion of the database 5 which is secured to prevent others, except for the system operator, from accessing the database 5.

In addition to comprising a search function, the IMS 7 comprises a relationship determining function. For example, in response to a query and/or the user inputting information regarding a tissue into the tissue information system 1, the IMS 7 searches the database 5 and classifies tissue information within the database 5 by type or attribute (e.g., patient sex, age, disease, exposure to drugs, tissue/cell type, DSM-IV classification, cause of death, and the like), and/or by codes, such as by SNOMED® codes, ICD-9 codes, and/or DSM-IV-TR codes. In one embodiment, when all attributes have been defined and classified as characteristic of defined relationship(s), the IMS 7 assigns a relationship identification number to each attribute, or set of attributes, and signals representing these attribute(s) are stored in the database 5 (e.g., as part of the data dictionary subdatabase) where they are indexed by the relationship ID# and provided with a descriptor. For example, in one embodiment, the expression of a plurality of biological characteristics which have been classified as correlating to a neuropsychiatric state X (e.g., autism) is assigned an ID# and a descriptor such as “diagnostic traits of disease state X.” In a preferred embodiment, the relationship determining function of the IMS 7 relates psychological profiles which are indexed according to a patient's DSM-IV classification, with physiological profiles and/or molecular profiles (e.g., gene expression data) and/or behavioral profiles.

The relationship determining function of the IMS 7 can employ one or more statistical programs to identify groups of attributes which represent particular relationships. In one embodiment, the statistical program is a non-hierarchical clustering program. In another embodiment, the clustering program employs k-means clustering.

The IMS 7 analyzes the relationships between data in the database 5 and/or new data being inputted, using any method standardly used in the art, including, but not limited to, regression, decision trees, neural networks, fuzzy logic, and combinations thereof. In response to the results of this analysis, upon a query by a user, the system 1 displays at least one relationship or identifies that no discernable relationship can be found on the interface 6 of the user device 3. In one embodiment, the system 1 displays descriptors relating to plurality of relationships identified by the IMS 7 on the interface 6 as well as information relating to the statistical probability that a given relationship exists.

In one aspect, the user selects among a plurality of relationships identified by the IMS 7 by interfacing with the interface 6 to determine those of interest (e.g., a relationship between neuropsychiatric disease and the expression of a gene product might be of interest, while a relationship regarding hair color and a gene product might not be). In another embodiment of the invention, rather than scanning an entire database 5, the IMS 7 samples the database 5 randomly until at least one statistically satisfactory relationship is identified, with the user setting parameters for what is “statistically satisfactory.” In a further embodiment of the invention, the user identifies particular subdatabases for the IMS 7 to search. In still another embodiment, the IMS 7 itself identifies particular subdatabases based on query terms the user of the system 1 has provided.

In one aspect, the IMS-7 is used to identify populations of patients who share selected clinical characteristics by identifying sources of tissue samples who have these clinical characteristics. Clinical characteristics may be embodied in data which have already been entered into the database 5 or may be embodied in new data, which is being inputted into the system for validation. In one embodiment, populations of patients are identified who share a particular clinical history or outcome, a specific type of physiological response to a drug, either adverse or beneficial, or a specific behavioral characteristics (e.g., depression).

In one aspect, a relationship identified by the IMS 7 is used to identify diagnostic traits associated with a particular neuropsychiatric disorder. For example, where a relationship identified indicates a high correlation between a neuropsychiatric disorder and the expression of one or more biological characteristics in tissue samples from a patient, the expression of the one or more biological characteristics can then be used to identify the presence of the disorder in other patients. For example, the relationship determining function of the IMS-7 (e.g., an application program which performs k-means clustering) can be used to designate potential pathway genes which are expressed during a neuropsychiatric and whose expression is related to the expression of other genes in the pathway.

Thus, in a very simple embodiment, where a schizophrenic patient A expresses genes 1, 2, 3, 4, a schizophrenic patient B expresses genes 1, 2, 4, 7, 8, a schizophrenic patient C expresses genes 1, 2, 4, 8, 9, 10, and normal patients D, E, and F express genes 2, 3, 8, the IMS 7 would identify genes 1, 4, 7, 9, and 10 as potentially involved in a pathway altered in patients with schizophrenia and would rank genes 1 and 4 as being highly likely to be pathway genes involved in the pathology of schizophrenia. In a further embodiment, the IMS 7, in response to a user query would identify other patient parameters associated with the expression of genes 7, 9, and 10 and would perform clustering analyses to determine whether any relationships identified were statistically unlikely to arise by chance. For example, the IMS 7 might identify that patients expressing genes 7, 9, and 10, in addition to having schizophrenia, show a statistically significant likelihood of suffering from neurodegenerative diseases. The IMS 7 can also reveal correlations between demographic factors and particular neuropsychiatric disorders. For example, the relationship determining function of the IMS 7 might show that patients with disease X show a statistically significant tendency to reside within 50 miles certain types of industrial plants or sources of particular types of pollutants.

In a preferred aspect of the invention, the IMS 7 includes an expert system. For example, the IMS 7 can comprise an object-oriented deployment system (e.g., such as the G2 Version 3.0 Real Time Expert System, available from Gensym, Corp.). Static Expert systems can also be used. Expert systems can be used to establish rules and procedures to identify and validate molecular pathways and to correlate changes in the expression of pathway biomolecules with any of the physiological responses described above. In one aspect, the expert system includes an inference function that operates on information within the specimen-linked database 5 and its associated subdatabases to identify biomolecules which are likely to belong to a pathway. The inference function allows the system 1 to rank pathways identified according to their probability of occurrence given the information which has been inputted into the database 5. In other aspects, the system 1 can be directed by a user to simulate pathways and to compare these pathways with molecular profiling data within the database 5. Preferably, the IMS 7 ranks simulated pathways according to their likelihood of occurrence based on data obtained from a plurality of tissue microarrays. The expert system of the IMS 7 can further include a transaction manager whose function is to direct input and output requests between one or more servers 4 of the system 1 and the interfaces of one or more user devices 3 of the system, in order to respond to user requests.

Expert systems are known in the art and include such systems as MYCIN, EMYCIN, NEOMYCIN, and HERACLES (see, e.g., Clancy, August, 1986, The AI Magazine pp. 40-60; Thompson et al., 1986, IEEE Software, pp. 6-15; Bylander, August, 1986, The AI Magazine, pp. 66-77; Hofmann et al., 1986, Expert Systems, 3(1): 4-11; and Yung-Choa Pan et al., Fall, 1986, The AI Magazine, pp. 62-69). Other expert systems are described in, for example, U.S. Pat. No. 6,154,750, U.S. Pat. No. 6,188,988, U.S. Pat. No. 6,149,585, U.S. Pat. No. 6,055,507, U.S. Pat. No. 5,991,730, and U.S. Pat. No. 5,777,888, and U.S. Pat. No. 4,866,635. The entireties of these references are incorporated by reference herein.

Relationships identified by the IMS 7 can be displayed to the user in a variety of formats such as graphs, histograms, dendograms, charts, tables and the like. In a preferred embodiment, in response to a request by a user, the system 1 displays on the interface 6 of a user device 3 a representation of a molecular pathway which includes a plurality of pathway biomolecules graphically arranged according to their effect on the expression of other pathway biomolecules (e.g., connected by arrows and the like). When a user selects a particular pathway biomolecule on the “pathway interface” (e.g., by moving a cursor to a representation of the biomolecule, such as the biomolecule's name), the user is linked to an interface which provides information relating to the biomolecule. The interface can alternatively, or additionally, provide information category links which provide the user with access to portions of the database 5 which comprise information related to a particular information category.

Information about a biomolecule can include a three-dimensional molecular structure information, sequence information and/or links to external genomic and/or protein databases, where appropriate (e.g., such as GenBank or SWISS-Prot), information relating to one or more of: mutations, allelic variants, ligands, substrates, products, cofactors, agonists, and antagonists, reference links to external databases including references about the biomolecule (e.g., PubMed), and information about available clones (e.g., cDNA molecules expressing a pathway protein), if applicable, and the like.

In a preferred embodiment, the user can access an “expression profile interface” on which is displayed a representation of the levels and/or forms of expression of the selected pathway biomolecule in a plurality of tissues. Preferably, this interface is also associated with one or more information category links identifying physiological response categories such as responses to diseases, pathological conditions, drugs or other agents, environmental conditions and the like. Selecting one of these information categories will link the user to an interface on which is displayed an expression profile of the biomolecule during a particular physiological response. In certain embodiments, the expression profiles of pathway molecules in a plurality of tissues during a plurality of different physiological responses is displayed on a single interface for comparison. In one embodiment, in response to a user query, the system performs an electronic subtraction analysis and displays differences in expression profiles on a single interface. Electronic subtraction methods are known in the art (see, for example, U.S. Pat. No. 6,114,114, the entirety of which is incorporated by reference herein). A “pathway home” button can be provided on any or all of these interfaces to direct a user back to the interface displaying the pathway.

In one aspect, selecting a pathway biomolecule on a pathway interface provided by the system 1 displays a pull down menu which provides the user with the simulation options, such as“delete,” “underexpress” and/or “overexpress.” Selecting one of these options directs the IMS 7 to simulate the effects of deleting, underexpressing and/or overexpressing the biomolecule identified on the expression of other biomolecules in the pathway. In some embodiments, selecting “underexpress” or “overexpress” causes a pull down menu of values to be displayed (e.g., 2× or −2×; selecting 2× would show the effects of doubling the biomolecule, while selecting −2× would show the effects of halving the biomolecule). In some embodiments, the system 1 is used to model the effect of one or more feedback loops on the pathway.

In some aspects, selecting a representation of a receptor in a pathway interface (e.g., such as a GPCR) links the user to an interface which displays information categories links relating to “antagonists” and “agonists” of the receptor molecule. These links provide a user with access to portions of the specimen-linked database which include information relating to molecules which have been demonstrated to alter the interaction of the receptor with its ligand. These molecules can include drugs with known dissociation constants and characterized circulating half lives. However, in other embodiments, the user can direct the IMS 7 to simulate the molecular structure of antagonist or agonist molecule and model the effect of binding such a molecule to the receptor on the expression of other pathway molecules in the pathway to which the receptor belongs. In silico modeling of receptor ligand interactions is known in the art and is described in, for example, Lengauer et al., 1996, Curr. Opin. Struct. Biol. 5: 402-406; Strynadka et al., 1996, Nature Struct. Bio. 3: 233-239; Chen et al., 1997, Biochemistry 36: 11402-11407 (1997); and Kuntz et al., J. Mol. Biol. 161: 269-288 (1982); the entireties of which are incorporated by reference herein.

In some aspects, the IMS 7 is used to identify the effects of agents (e.g., antagonists or agonists or potentially toxic agents) on a plurality of pathway molecules by comparing the physiological responses of cells in culture exposed to one or more agents with the biological characteristics of samples of these cells arrayed on tissue microarrays. Thus, in some aspects, the IC₅₀ value, or the concentration of an agent that causes 50% growth inhibition, the GI₅₀ value (which measures the growth inhibitory effect of an agent) the TGI (which provides a measure of an agent's cytostatic effect), and/or the LC₅₀ (which provides a measure of the agent's cytotoxic effect) is measured in vitro and correlated with the expression of one or more pathway biomolecules in samples on microarrays. In the case of agonists or antagonists, the effects of these agents on dissociation constants and other kinetic parameters of biological receptors can also be measured.

In some embodiments, in response to a user query, the system 1 displays a “mean graph” interface or an interface which provides a display of the pattern created by plotting positive and negative values generated from a set of GI₅₀, TGI, or LC₅₀ values. For example, positive and negative values can be shown plotted along a vertical line that represents the mean response of all cells exposed to an agent. Positive values provide a measure of which cellular sensitivities are significant, while negative values indicate results that are not significant. Mean graphs are described in, for example, Paull et al., 1989, J. Natl. Cancer Inst. 81: 1088-1092;. Paull et al., 1988, Proc. Am. Assoc. Cancer Res. 29: 488, the entireties of which are incorporated by reference herein.

In some aspects, the IMS 7 implements a COMPARE algorithm to provide an ordered list of agents ranked according to their effects on the physiological responses of cells and/or tissues and on the expression of biomolecules in these cells and/or tissues. COMPARE algorithms are described in Paul et al., supra, and in Hodes et al., 1992, J. Biopharm. Stat. 2: 31-48, the entireties of which are incorporated by reference herein. Data obtained from this analysis can be added to the specimen-linked database 5 and made available to other users of the system 1. The IMS 7 also can include statistical programs to facilitate comparisons such as PROC CORR. Other algorithms, such as the DISCOVER algorithm also can be used.

In a preferred embodiment, in response to a user query, the system 1 will display an interface which includes a representation of the expression profiles of pathway biomolecules in tissues exposed to an agent characterized as described above. In still more preferred embodiments, the system 1 will perform an electronic subtraction to show only changes in expression profiles in treated tissues compared to untreated tissues. In still other embodiments, changes in expression values are expressed as ratios of differences (e.g., level of biomolecule A in treated tissue 1/level of biomolecule A in untreated tissue 1) or as percent changes of expression.

The above assays can be performed in parallel with assays using animals who have also been exposed to the same agents to compare the physiological responses of these animals with the expression of pathway biomolecules in whole body tissue microarrays obtained from these animals. Preferably, the animals are models of neuropsychiatric diseases or aberrant behavioral responses (e.g., high levels of aggression). Physiological responses measured can include the overall health of the animal, organ function, levels of metabolites and other molecules in the blood, and the like. In some embodiments, the localization of the agents in tissues on the microarrays is determined, for example, by using labeled aptamer probes or other molecular probes which recognize these agents. Preferably, the above assays are also performed with assays to evaluate the behavior of the animal at various time points after exposure to an agent.

Similarly, the physiological responses of patients to agents can also be correlated with the expression of a plurality of pathway biomolecules by using tissue microarrays. In some aspects, patient samples are derived from autopsies and the expression of pathway biomolecules in whole body tissue microarrays is correlated with detailed information relating to the patient's medical history (e.g., including drug exposure), psychological evaluations of the patient by one or more health care workers, family medical history, and other characteristics which have been inputted into the specimen-linked database 5.

In one aspect, the system 1 provides treatment information, such as medication recommendations, health care provider information, and the like, that have been demonstrated as being successful (associated with a greater than 20%, and preferably greater than 50% amelioration of symptoms) in treating patients with similar behavioral profiles, physiological profiles, and/or molecular profiles. Additionally, the system 1 can provide information about treatment options which are currently under investigation (e.g., such as clinical trial information) In another aspect, a user of the system is provided with contact information (e.g., such as an email address) of a health care provider (e.g., a psychiatrist, a physician, a psychologist, a licensed social worker) and can provide the health care provider with permission to access portions of the database comprising information associated with particular patient(s). Information about the provider can include age, sex, licenses held, address, phone number, areas of treatment expertise, affiliations (e.g., with particular insurance plans or HMOs) and the like.

In one aspect, the user is able to view, print, permanently store, read, and/or further manipulate data displayed on the display 6 of his or her device 3. In this embodiment, the user is able to use the system 1 to investigate and define the relationships most relevant to tissues or diseases of interest. In one embodiment, the user is also able to link to any database publicly accessible through the network 2, and to integrate information from such a database with the system 1's database 5 through the IMS 7. Thus, in one embodiment, information can be shared with other users and information from other users can be continuously added to the database 5.

One embodiment of the invention recognizes potential difficulties in enabling unrestricted access to the database 5, and encompasses providing restricted access to the database 5, and/or restricted ability to change the contents of the database 5 or records in the database 5 using the IMS 7 and/or a security application. Methods of providing restricted access to electronic data are known in the art, and are described, for example, in U.S. Pat. No. 5,910,987, the entirety of which is incorporated by reference herein.

Molecular Probes

Antibodies for Detection of Biological Characteristics

Antibodies specific for a large number of known antigens are commercially available. Links to multiple antibody suppliers can also be found at http:// www.antibodyresource.com/misc.html. When antibodies are not commercially available, one of skill in the art can readily raise their own antibodies using standard techniques.

In order to produce antibodies, various host animals are immunized by injection with the growth-related polypeptide or an antigenic fragment thereof. Useful animals include, but are not limited to rabbits, mice, rats, goats, and sheep. Adjuvants may be used to increase the immunological response to the antigen. Examples include, but are not limited to, Freund's adjuvant (complete and incomplete), mineral gels such as aluminum hydroxide, surface active substances such as lysolecithin, pluronic polyols, polyanions, peptides, oil emulsions, keyhole limpet hemocyanin, dinitrophenol, and adjuvants useful in humans, such as BCG (bacille Calmette-Guerin) and Corynebacterium parvum. These approaches will generate polyclonal antibodies.

Monoclonal antibodies specific for a polypeptide may be prepared using any technique that provides for the production of antibody molecules by continuous cell lines in culture. These include, but are not limited to, the hybridoma technique originally described by Kohler and Milstein, 1975, Nature 256: 495-497; the human B-cell hybridoma technique (Kosbor et al., 1983, Immunology Today 4: 72; Cote et al., 1983, Proc. Natl. Acad. Sci. USA. 80: 2026-2030) and the EBV-hybridoma technique (Cole et al., 1885, In Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci. USA 81: 6851-6855); Neuberger et al., 1984, Nature 312: 604-608; Takeda et al., 1985, Nature 314: 452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce growth-related polypeptide-specific single chain antibodies. The entireties of these references are incorporated by reference herein.

Antibody fragments which contain specific binding sites of a growth-related polypeptide may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al., 1989, Science 246: 1275-1281) to allow rapid and easy identification of monoclonal Fab fragments with the desired specificity to a growth-related polypeptide. An advantage of cloned Fab fragment genes is that it is a straightforward process to generate fusion proteins with, for example, green fluorescent protein for labeling.

Antibodies, or fragments of antibodies may be used to quantitatively or qualitatively detect the presence of growth-related polypeptides or conserved variants or peptide fragments thereof. For example, immunofluorescence techniques employing a fluorescently labeled antibody coupled with light microscopic, or fluorimetric detection can be used.

Antibodies or antigen binding portions thereof may be employed histologically, as in immunohistochemistry, immunofluorescence, immunoelectron microscopy, or an histological assays, for in situ detection of polypeptides or other antigen-containing biomolecules.

Allele-Specific Antibodies and Modification-Specific Antibodies

In preferred embodiments, antibodies are used which are specific for specific allelic variants of a protein or which can distinguish the modified from the unmodified form of a protein (e.g., such as a phosphorylated vs. an unphosphorylated form or a glycosylated vs. an unglycosylated form of a polypeptide, adenosylated vs. unadenosylated forms of a polypeptide). For example, peptides comprising protein allelic variations can be used as antigens to screen for antibodies specific for these variants. Similarly modified peptides or proteins can be used as immunogens to select antibodies which bind only to the modified form of the protein and not to the unmodified form. Methods of making allele-specific antibodies and modification-specific antibodies are known in the art and described in U.S. Pat. No. 6,054,273; U.S. Pat. No. 6,054,273; U.S. Pat. No. 6,037,135; U.S. Pat. No. 6,022,683; U.S. Pat. No. 5,702,890; U.S. Pat. No. 5,702,890, and in Sutton et al., J. Immunogenet. 14(1): 43-57 (1987), the entireties of which are incorporated by reference herein.

Immunohistochemistry (IHC)

In situ detection of an antigen can be accomplished by contacting a test tissue and microarray on a profile array substrate with a labeled antibody that specifically binds the antigen. The antibody or antigen binding portion thereof is preferably applied by overlaying the labeled antibody or antigen binding portion onto the test tissue and microarray. Through the use of such a procedure, it is possible to determine not only the presence of the antigen but also its amount and its localization in a test tissue and in the plurality of sublocations within the microarray.

In one embodiment, antibodies are detectably labeled by linkage to an enzyme for use in an enzyme immunoassay (EIA) (Voller 1978, Diagnostic Horizons 2: 1-7; Voller et al., J. Clin. Pathol. 31: 507-520 (1978); Butler, 1981, Meth. Enzymol. 73: 482-523). The enzyme which is linked to the antibody will react with an appropriate substrate, preferably a chromogenic substrate, in such a manner as to produce a chemical moiety which is detectable, for example, by spectrophotometric, fluorimetric or visual means. Examples of enzymes useful in the methods of the invention include, but are not limited to peroxidase, alkaline phosphatase, and RTU AEC.

Detection of bound antibodies can alternatively be performed by radiolabeling antibodies and detecting the radiolabel. Following binding of the antibodies and washing, the samples may be processed for autoradiography to permit the detection of label on particular cells in the samples.

In one embodiment, antibodies are labeled with a fluorescent compound. When the fluorescently labeled antibody is exposed to light of the proper wavelength, its presence can be detected due to fluorescence. Many fluorescent labels are known in the art and may be used in the methods of the invention. Preferred fluorescent labels include fluorescein, amino coumarin acetic acid, tetramethylrhodamine isothiocyanate (TRITC), Texas Red, Cy3.0 and Cy5.0. Green fluorescent protein (GFP) is also useful for fluorescent labeling, and can be used to label non-antibody protein probes as well as antibodies or antigen binding fragments thereof by expression as fusion proteins. GFP-encoding vectors designed for the creation of fusion proteins are commercially available.

The primary antibody (the one specific for the antigen of interest) may alternatively be unlabeled, with detection based upon subsequent reaction of bound primary antibody with a detectably labeled secondary antibody specific for the primary antibody. Another alternative to labeling of the primary or secondary antibody is to label the antibody with one member of a specific binding pair. Following binding of the antibody-binding pair member complex to the sample, the other member of the specific binding pair, having a fluorescent or other label, is added. The interaction of the two partners of the specific binding pair results in binding the detectable label to the site of primary antibody binding, thereby allowing detection. Specific binding pairs useful in the methods of the invention include, for example, biotin:avidin. A related labeling and detection scheme is to label the primary antibody with another antigen, such as digoxigenin. Following binding of the antigen-labeled antibody to the sample, detectably labeled secondary antibody specific for the labeling antigen, for example, anti-digoxigenin antibody, is added which binds to the antigen-labeled antibody, permitting detection.

The staining of tissues for antibody detection is well known in the art, and can be performed with molecular probes including, but not limited to, AP-Labeled Affinity Purified Antibodies, FITC-Labeled Secondary Antibodies, Biotin-HRP Conjugate, Avidin-HRP Conjugate, Avidin-Colloidal Gold, Super-Low-Noise Avidin, Colloidal Gold, ABC Immu Detect, Lab Immunodetect, DAB Stain, ACE Stain, NI-DAB Stain, polyclonal secondary antibodies, biotinylated affinity purified antibodies, HRP-labeled affinity purified antibodies, and/or conjugated antibodies.

In one embodiment, immunohistochemistry is performed using an automated system such as the Ventana ES System and Ventana gen^{II}™ System (Ventana Medical Systems, Inc., Tucson, Ariz.). Methods of using this system are described in U.S. Pat. No. 5,225,325, U.S. Pat. No. 5,232,664, U.S. Pat. No. 5,322,771, U.S. Pat. No. 5,418,138, and U.S. Pat. No. 5,432,056, the entireties of which are incorporated by reference herein.

Nucleic Acid Probes

Nucleic acid probes can also be used where the sequence of a gene encoding a biomolecule is known. Means for detecting specific DNA sequences within genes are well known to those of skill in the art. In one embodiment, oligonucleotide probes chosen to be complementary to a selected subsequence within the gene can be used. Nucleic acid probes can be fragments of larger nucleic acid molecules (e.g., such as obtained by restriction enzyme digestion or by PCR or another amplification technique) or can be synthetic molecules. Modified nucleic acids (e.g., comprising one or more altered bases, sugars, and/or internucleotide linkages) and analogs (e.g., such as PNA molecules) are also encompassed within the scope of the invention.

Methods of labeling nucleic acids are well known to those of skill in the art. Preferred labels are those that are suitable for use in in situ hybridization (ISH) or fluorescent in situ hybridization (FISH). In one embodiment, nucleic acid probes are detectably labeled prior to hybridization with a tissue sample. Alternatively, a detectable label which binds to the hybridization product can be used. Labels for nucleic acid probes include any composition detectable by spectroscopic, photochemical, biochemical, immunochemical, or chemical means and include, but are not limited to, radioactive labels (e.g. ³²P, ¹²⁵I, ¹⁴C, ³H, and ³⁵S), fluorescent dyes (e.g. fluorescein, rhodamine, Texas Red, etc.), electron-dense reagents (e.g. gold), enzymes (as commonly used in an ELISA), colorimetric labels (e.g. colloidal gold), magnetic labels (e.g. Dynabeads TM), and the like. Examples of labels which are not directly detected but are detected through the use of directly detectable label include biotin and dioxigenin as well as haptens and proteins for which labeled antisera or monoclonal antibodies are available.

A direct labeled probe, as used herein, is a probe to which a detectable label is attached. Because the direct label is already attached to the probe, no subsequent steps are required to associate the probe with the detectable label. In contrast, an indirect labeled probe is one which bears a moiety to which a detectable label is subsequently bound, typically after the probe is hybridized with the target nucleic acid.

Labels can be coupled to nucleic acid probes in a variety of means known to those of skill in the art. In some embodiments the nucleic acid probes are labeled using nick translation or random primer extension (Rigby et al. 1977, J. Mol. Biol., 113: 237 or Sambrook et al., Molecular Cloning: A Laboratory Manual, Cold Spring Harbor Laboratory, Cold Spring Harbor, N.Y., (1989), the entireties of which are incorporated by reference herein).

Alternatively, sequences or subsequences of tissues within a microarray may be amplified by a variety of DNA amplification techniques (e.g., polymerase chain reaction, ligase chain reaction, transcription amplification, etc.) prior to detection using a probe. Amplification of nucleic acid sequences increases sensitivity by providing more copies of possible target subsequences. In addition, by using labeled primers in the amplification process, the sequences are labeled as they are amplified.

Aptamer Probes

Aptamer probes are also encompassed within the scope of the invention, e.g., to label molecules which are not readily bound by nucleic acids using Watson-Crick binding or by antibodies. Methods of generating aptamers are known in the art and described in U.S. Pat. No. 6,180,406 and U.S. Pat. No. 6,051,388, for example, the entireties of which are incorporated by reference herein. Aptamers can generally be labeled as described above with reference to nucleic acid probes.

In situ Hybridization (ISH) and Fluorescent in situ Hybridization (FISH)

In situ hybridization (ISH) and Fluorescent In Situ Hybridization (FISH) are techniques that can avail themselves to paraffin-embedded sectioned tissue. Both techniques are genomic based rather than proteomic based, as in IHC, and involve RNA and DNA probes that will hybridize, or specifically bind to their complement base sequence. In some embodiments, labels are attached to genomic probes that allow hybridization of the probes to be visualized under a microscope. ISH probes generally have a chromogenic marker and can be observed by traditional light microscopy. FISH probes generally have a fluorescent marker bonded and must be visualized with the use of a fluorescent microscope.

In one embodiment, for in situ hybridization of paraffin-embedded tissues, sections of paraffin-embedded tissue immobilized on glass substrates are treated as follows: substrates are dewaxed in staining dishes by three changes in xylene for 2 minutes each (dewaxing is not necessary for non-embedded single cells); dewaxed samples are then rehydrated using the following procedure: exposure to 100% ethanol, two times for two minutes, then subsequent 2 minute incubations in 95%, 70%, and 50% ethanol. (It should be apparent to those of ordinary skill in the art that the incubation time is not critical and may be optimized, but in general should be at least two minutes.) Samples are denatured (e.g., by incubation for 20 minutes at room temperature in 0.2 N HCl, followed by heat denaturation for 15 minutes at 70° C. in 2× SSC). Samples are then rinsed, for example, in 1× PBS for 2 minutes. In some situations, usually empirically determined, a pronase digestion step may be included here which later allows improved access of the probes to the nucleic acids contained within the tissue sections. In such cases, samples are digested for 15 minutes at 37° C. with pre-digested, lyophilized pronase at an empirically determined concentration which allows hybridization yet preserves the cellular morphology (e.g., such as 0.1 to 10 μg/ml).

Pronase-digested samples are incubated for 30 seconds in a wash buffer, such as 2 mg/ml glycine in 1× PBS, to stop the digestion process. Samples may be post-fixed, for example, using freshly prepared 4% paraformaldehyde in 1× PBS, for 5 minutes at room temperature. Fixation is stopped by further washes, e.g., a 5 minute incubation in 3× PBS, followed by two 30 second rinses in 1× PBS. Samples are then soaked in 10 mM DTT, 1× PBS, for 10 minutes at 45° C., followed by a 2 minute incubation in 0.1 M triethanolamine, pH 8.0 (triethanolamine buffer). Next, samples are placed in fresh triethanolamine buffer to which acetic anhydride is added to 0.25% final concentration, followed by mixing and 5 minutes' incubation with gentle agitation. In one embodiment, more acetic anhydride is added to a final concentration of 0.5%, followed by 5 minutes' further incubation. Samples are washed, for example, for 5 minutes in 2× SSC, and by dehydrated by successive incubation in 50%, 70%, 95% and 100% ethanol for 2 minutes each at room temperature. Preferably, samples are air-dried or dried with desiccant before proceeding to the hybridization step. Any, or all, of the preceding series of steps may be automated in order to increase throughput.

Probes for in situ hybridization may be DNA or RNA oligonucleotides (e.g., RNA transcribed in vitro). In one embodiment, RNA probes labeled with ³⁵S are dissolved in 50 mM dithiothreitol (DTT) and are added to a non-specific competitor. In one embodiment, the competitor is preferably RNA made in the same manner as the labeled specific probe, except from a transcription template with non-specific sequences, such as a vector with no insert. No labeled ribonucleosides are in the reaction mix.

The probe/non-specific competitor mixture is then denatured, for example, by heating at 100° C. for 3 minutes, and added to a hybridization buffer (e.g., such as 50% (v/v) deionized formamide, 0.3 M NaCl, 10 mM Tris (pH 8.0), 1 mM EDTA, 1× Denhardt's solution, 500 mg/ml yeast tRNA, 500 mg/ml poly(A), 50 mM DTT, and 10% polyethylene glycol 6000) to a 0.3 μg/ml-10 μg/ml final probe concentration. An estimate of the amount of probe synthesized is based on a calculation of the percent of the label incorporated and the proportion of the labeled base in the probe molecule as a whole. In one embodiment, the non-specific competitor is provided in an amount approximately equal to one half the mass of labeled probe.

The probe/hybridization mix is incubated at 45° C. until applied to the microarrays and test tissue sample as a thin layer of liquid. Hybridization reactions are generally incubated in a moist chamber such as a closed container containing towels moistened with 50% deionized formamide, 0.3 M NaCl, 10 mM Tris (pH 8.0), 1 mM EDTA, at 45° C. If background (e.g., the amount of non-specific labeling) proves to be a problem, a 1 to 2 hour pre-hybridization step using only non-specific, unlabeled riboprobe competitor in hybridization buffer can be added prior to the step in which labeled probe is applied.

In one embodiment, hybridization is carried out for 30 minutes to 4 hours, followed by washing to remove any unbound probe. In one embodiment, the profile array substrates are washed in an excess (100 ml each wash) of the following buffers: 50% formamide, 2× SSC, 20 mM β-mercaptoethanol, two times, for 15 minutes at 55° C.; 50% formamide, 2× SSC, 20 mM β-mercaptoethanol, 0.5% Triton X-100, two times, for 15 minutes at 55° C.; and 2× SSC, 20 mM β-mercaptoethanol, two times, for 2 minutes at 50° C.

In another embodiment, samples are subjected to RNAse digestion for 15 minutes at room temperature for example using a solution containing 40 mg/ml RNase A, 2 mg/ml RNase T1, 10 mM Tris (pH 7.5), 5 mM EDTA and 0.3 M NaCl. In one embodiment, after RNase digestion, slides are soaked two times for 30 minutes each in 2× SSC, 20 mM β-mercaptoethanol at 50° C., followed by two washes in 50% formamide, 2× SSC, 20 mM β-mercaptoethanol at 50° C. and two washes of 5 minutes each in 2× SSC at room temperature. Hybridized, washed slides are dehydrated through successive two minute incubations in the following: 50% ethanol, 0.3 M ammonium acetate; 70% ethanol, 0.3 M ammonium acetate; 95% ethanol, 0.3 M ammonium acetate; 100% ethanol. Slides are air dried overnight and with emulsion for autoradiography according to standard methods.

Sections prepared from frozen tissues may be hybridized by a similar method except that the dewaxing and paraformaldehyde fixation steps are omitted. For details, see Ausubel et al., 1992, Short Protocols in Molecular Biology, (John Wiley and Sons, Inc.), pp. 14-15 to 14-16, the entirety of which is incorporated by reference herein. In still another embodiment, ISH or FISH is performed with one or more amplification steps, i.e., such as by performing in situ PCR. A detailed description of these techniques are presented in Ausubel, et al., 1992, supra, pp. 14-37 to 14-49, the contents of which are hereby incorporated by reference.

In a further embodiment of the invention, information obtained from a single sublocation on a microarray can be information relative to the expression of both proteins and nucleic acids. For example, in one embodiment of the invention, after performing immunohistochemistry on tissue at a sublocation, a portion of the tissue is obtained to isolate nucleic acids which are further analyzed by amplification methods such as PCR. Detection of nucleic acids isolated from an embedded tissue sample is known in the art and is described in, for example, U.S. Pat. No. 6,013,461, U.S. Pat. No. 6,110,902, and U.S. Pat. No. 6,114, 110, the entireties of which are incorporated by reference herein.

In still a further embodiment, tissues can be counterstained to highlight their morphology (e.g., with hematoxylin/eosin, or one or more combination of other dyes, such as described in Ausubel et al., 1992, supra, pp. 14-19 to 14-22).

As with the IHC techniques described above, nucleic acid hybridization techniques can also be automated. In one embodiment, both detection and probing is automated. For example, in one embodiment, a profile array substrate which has been, or is being reacted, with a molecular probe is in communication with a detector. A light source in proximity to the tissue samples on the substrate transmits light to the samples and light transmitted by the samples is received by the detector. In one embodiment, the detector is in communication with the tissue information system described above and signals transmitted to the tissue information system relating to optical information from the tissues are displayed and/or stored within the electronic database. In one embodiment, optical information from tissue samples on the microarray is displayed as an image of tissue(s) on the interface of the display of a user device included in the tissue information system.

Kits

The invention further provides kits. A kit according to the invention, minimally contains a tissue microarray 13 and provides access to an information database (e.g., in the form of a URL and an identifier which identifies the particular microarray being used, and/or a password). In one embodiment, the kit comprises instructions for accessing the database 5, or one or more molecular probes, for obtaining molecular profiling data using the microarray 13, and/or other reagents necessary for performing molecular profiling (e.g., labels, suitable buffers, and the like). In a preferred embodiment, kits are provided which include a panel of molecular probes reactive with a plurality of pathway biomolecules.

### EXAMPLES

The invention will now be further illustrated with reference to the following examples. It will be appreciated that what follows is by way of example only and that modifications to detail may be made while still falling within the scope of the invention.

### Example 1

Blood is collected from a plurality of patients classified as having a specific neuropsychiatric disorder using DSM-IV criteria. Blood cells are processed to generate donor blocks as described above for the generation of microarrays, with or without a purification step (e.g., such as flow cytometry or ficoll hypaque density gradient centrifugation) to enrich for lymphocytes, for example. Blood cells from normal patients sharing similar demographic traits as the patients having the neuropsychiatric disorder are also collected and used to generate microarrays. Control samples can be arrayed on the same or different microarrays are the test samples. Samples are also obtained comprising neural tissue samples from autopsies and/or other pathology procedures from patients who have been diagnosed according to the same DSM-IV criteria and from demographically matched normal patients. These samples can be arrayed on the same or different substrates as the blood cell samples.

The microarrays are then contacted with at least one molecular probe and preferably with a plurality of molecular probes (simultaneously or sequentially) and gene expression data is determined. Molecular probes can be probes which react specifically with any of the pathway molecules identified above or can be probes which react with sequences from uncharacterized genes (e.g., EST probes and/or SNP probes), or genes which generally expressed in neural tissues, but for which the relationship to other pathway molecules is not known. Information relating to the reactivity of the probe(s) with the microarrays is determined and is inputted into the system 1 by a user using a user device 3 and the IMS 7 is prompted by the user to perform an electronic subtraction analysis to identify differentially expressed genes (see, e.g., as described in U.S. Pat. No. 6,114,114, the entirety of which is incorporated by reference herein).

In a preferred embodiment, differentially expressed genes whose expression is correlated with the DSM-IV classification of the patient are identified. Such genes are further ranked according to whether they are differentially expressed in both neural tissues and tissues from blood cells of patients. In a preferred aspect, a gene which is differentially expressed in both neural tissues and blood cells is identified as a candidate marker for a specific DSM IV category disease. Preferably, patient information is collected both from living patients and from the autopsy patients and added to the database. Markers can further be characterized using the IMS 7 according to demographic traits of the patients from whom the samples have been obtained (e.g., age, sex, presence of other diseases, and the like).

### Example 2

In one aspect, microarrays are generated which comprise one or more samples from living patients (e.g., such as blood cell samples) and reacted with one or more molecular probes as described above. The patients from whom samples have been obtained have also been administered a radiolabeled ligand which binds to a neurotransmitter receptor, such as are known in the art. The distribution and quantity of the ligands binding to cells in the brain is determined using positron emission tomography (or PET) (see, e.g., as described in Farde et al., 1997, Nature 385: 590) and provides a measure of the amount/density of receptors for the neurotransmitter. This measure is provided to the system 1, and information relating to this measure is stored in the database 5 and is correlated with information relating to the reactivity of the molecular probes by the IMS 7. In this way, the system 1 is used to identify relationships between a neuropsychiatric disorder, the level and/or density of particular neurotransmitter receptors, and the expression and/or localization of biomolecules which react with the one or more molecular probes in tissues/cells from a patient. Preferably, the patients are diagnosed as having one or more neuropsychiatric disorders using DSM-IV criteria, and this information is also inputted into the system 1 using DSM-IV-TR codes to index records from these patients as described above.

### Example 3

In one aspect, samples from a plurality of schizophrenic patients are arrayed on a microarray and assayed for the presence or absence of an adenosylated D4 receptor using antibodies which specifically bind to the adenosylated form and not to the non-adenosylated form of the receptor (see, e.g., WO 96/37780). Identical arrays (e.g., generated from the same recipient block, and preferably from sections within 50-100 μm of each other in a recipient block) are probed with antibodies which specifically bind the non-adenosylated form and/or with antibodies which recognize both forms of the receptor. Blood cell samples (e.g., lymphocytes) can be used for this type of assay, and in one embodiment, it is contemplated that samples from living patients are obtained. Preferably arrays are also probed with molecular probes reactive with one or more of dopamine, methionine adenosyltransferase (MAT), phospholipid methyltransferase I, phospholipid methyltransferase II, methylated phospholipids (e.g., such as methylated phosphatidylethanolamine (PE)), adenosylhomocysteine hydrolase, methionine synthase, serine hydroxymethyltransferase, Catechol-O-methyltransferase (COMT), and other D4 pathway gene products. Additional microarrays can also be evaluated for the expression other dopamine pathway biomolecules (e.g., D1, D2, D3 and D5 pathway molecules. Microarrays are preferably reacted with both RNA-reactive probes (e.g., labeled DNA probes or primers which specifically bind to dopamine pathway transcripts and protein-reactive probes (e.g., antibodies). For example, identical microarrays can be reacted in parallel to determine the expression of RNA as well as protein products of dopamine pathway genes. In one aspect, nucleic acid samples are simultaneously obtained from patients who have provided samples for the microarrays, and RT-PCR assays are performed on these samples using primers which specifically hybridize to one or more dopamine pathway receptor transcripts. Information relating to expression of such molecules is stored in the database 5 of the system 1.

### Example 4

Expressed sequences which are expressed in neural tissues are obtained from known expressed sequence databases (e.g., such as EST databases, or cDNA databases) and are used to generate nucleic acid microarrays using methods known in the art (see, e.g., as described in U.S. Pat. No. 6,183,968, for example, the entirety of which is incorporated by reference herein). Sets of identical arrays (i.e., arraying the same sequences) are contacted with labeled nucleic acids from bodily fluids of test patients afflicted with a neuropsychiatric disease and with labeled nucleic acids from control patients (e.g., patients with similar demographic characteristics but not having the disease) to identify nucleic acids which are differentially expressed in patients with the neuropsychiatric disease. The expression of these nucleic acids in both test and control microarrays is determined and compared to identify differentially expressed sequences in patients with the disease.

Differentially expressed sequences are then used as probes and reacted with neural tissues from patients with the same neuropsychiatric disease(s) (e.g., obtained from autopsy repositories comprising tissues from patients diagnosed as having the same disease using DSM-IV criteria and from demographically matched control patients not having the disease). Probes which are validated as being differentially expressed in neural tissues as well in these patients are then used in additional tests on microarrays comprising bodily fluid samples from populations of patients diagnosed with neuropsychiatric disease. Information relating to the reactivity of the probes with the arrays is stored in the database 5 and the IMS 7 is used to identify and rank probes which have high diagnostic utility (e.g., are significantly associated with the presence or absence of a neuropsychiatric disorder using routine statistical methods, and p values>0.005).

### Example 5

Samples from patients are evaluated using a plurality of different types of microarrays (e.g., at least two of: a tissue/cell microarray, a nucleic acid microarray, a protein/polypeptide/peptide microarray and the like). This approach can be exemplified with regard to the evaluation of physiological responses and gene expression in samples from patients presenting with characteristic features of trinucleotide repeat expansion (“TNR expansion”), i.e., diseases which demonstrate the phenomenon of anticipation, inheritance disposition (autosomal dominant and sex chromosomal dominant), neural regression or mental retardation, and somatic mosaicism. While normal patients will have tens of copies of TNRs in their genomic DNA, patients suffering from these diseases can carry up to hundreds as many times of these repeats. TNR expansion related diseases include, but are not limited to, spinocerebellar ataxia type III (SCA III), (see, U.S. Pat. No. 6,124,100, incorporated herein by reference), SCA I syndrome, SCA VI syndrome, SCA VII syndrome, FRAXE mental retardation, X-linked spinobular atrophy (SBMA), and dentatorubral and pallidoluysian atrophy (DRPLA).

Thus in one aspect, samples of nucleic acids (preferably, genomic DNA) are obtained from patients to test for TNR expansions, while tissue samples from the same patients are also obtained and arrayed on tissue/cell microarrays 13. Preferably, the presence of TNR repeats is quantified through the use of a nucleic acid array comprising probe oligonucleotides immobilized at a plurality of locations on a substrate (e.g., by spotting a nylon or nitrocellulose membrane or by immobilizing the probes in wells of a microtiter plate). Preferably, the probe comprises a portion of a gene comprising a TNR.

For example, in one embodiment, at least two types of probe are included at different locations on the substrate, i.e., a probe comprising a portion of the wild type SCA III gene comprising the 73 bp CAG repeat unit (e.g., comprising 13-34 copies of the TNR) and a probe comprising an a portion of an expanded SCA III gene (e.g., a sequence comprising 50 or more copies). Sample genomic DNA is hybridized to labeled primers capable of amplifying a portion of the SCA III gene comprising the repeat region and PCR products are hybridized to wild type SCA III gene probes and expanded gene probes, respectively. A sample which binds more to an expanded gene probe location than an unexpanded location is identified as a sample which comprises an expanded SCA III gene. In one aspect, primers are labeled with biotin and hybridization to the array substrate is detected by contacting the substrate with streptavidin-alkaline phosphatase and a chromogenic substrate. When the substrate is a microtiter plate, color formation can be quantitated by measuring absorbance (e.g., at 450 nm) using an automatic microtiter plate reader. This type of assay is described in U.S. Pat. No. 6,124,100. The presence/amount of repeat expansion is recorded and stored in the database 5 of the system 1.

Tissue/cell sample microarrays 13 comprising samples from the same patients are evaluated in parallel by reacting these microarrays 13 with one or more molecular probes reactive with one or more pathway molecules described above and/or or with molecular probes reactive with other neurally expressed gene products (characterized or uncharacterized). Expression data obtained from tissue/cell sample microarrays is then inputted into the system to provide a measure of physiological responses in the patients who provided the samples. Such responses are correlated with the presence/amount of repeat expansion observed in the nucleic acid arrays.

All literature citations, patents, and patent publications cited herein are incorporated by reference in their entirety. Variations and modifications of the above invention will be obvious to those of skill in the art and are encompassed within the instant invention.

What is claimed is:

1. An information system comprising:
a specimen-linked database comprising a plurality of information about a tissue microarray wherein the tissue microarray is identified by an identifier, the tissue microarray comprising a plurality of samples from at least one patient with a neuropsychiatric disorder;
at least one user device connectable to a network wherein the user device displays an interface wherein the plurality of information can be inputted into the interface; and
an information management system for searching the specimen-linked database and determining relationships between the plurality of information.

2. The information system of claim 1 further comprising a plurality of tissue microarrays.

3. The information system of claim 1 wherein the tissue microarray comprises a plurality of sublocations, wherein each sublocation being identified by a set of coordinates.

4. The information system of claim 3 wherein after the identifier is entered into the interface, the interface of the at least one user device displays a plurality of selectable coordinates wherein each set of coordinates corresponds to a set of coordinates for a single sublocation, wherein selection of the set of coordinates for a single sublocation causes the information management system to display a plurality of information about a sample located at the sublocation identified by the set of coordinates.

5. The information system of claim 4 wherein each set of coordinates is associated with a link for linking a user to the specimen-linked database.

6. The information system of claim 5 wherein the interface displays a plurality of information categories, wherein each information category is associated with a link to a portion of the specimen-linked database comprising information relating to the information category.

7. The information system of claim 3 wherein the interface of the user device displays a representation of the tissue microarray.

8. The information system of claim 7 wherein the representation of the tissue microarray comprises a plurality of images of samples at a plurality of sublocations on the tissue micro array.

9. The information system of claim 8 wherein each image is associated with a link for linking to the specimen-linked database.

10. The information system of claim 3 wherein the interface displays at least one field for inputting a set of coordinates of a sublocation of the tissue microarray.

11. The information system of claim 3 wherein the interface displays a plurality of information categories relating to a plurality of information available about a sample at a single sublocation.

12. The information system of claim 1 further comprising a plurality of records relating to an at least one physiological response of a plurality of patients having a neuropsychiatric disorder.

13. The information system of claim 12 wherein the plurality of records comprises a plurality of gene expression data.

14. The information system of claim 13 wherein the plurality of gene expression data comprises a plurality of data relating to an expression of a plurality of pathway biomolecules.

15. The information system of claim 14 wherein the plurality of pathway biomolecules comprise a plurality of biomolecules in a neurotransmitter receptor signalling pathway.

16. The information system of claim 15 wherein at least one of the plurality of biomolecules in the neurotransmitter receptor signaling pathway is selected from the group consisting of an adrenoreceptor, a dopamine receptor, an opioid receptor, cannabinoid receptor, a muscarinic receptor, a NMDA receptor, a mGlu receptor, a GABA receptor, a serotonin receptor, and combinations thereof.

17. The information system of claim 14 wherein the plurality of pathway biomolecules comprises a neurotransmitter, a neurotransmitter receptor, gene products involved in neurotransmitter synthesis, a neurotranmitter transporter, a G protein, and a kinase.

18. The information system of claim 12 wherein the neuropsychiatric disorder is classified using a DSM-IV criteria.

19. The information system of claim 12 wherein the plurality of records are indexed according to a DSM-IV classification of a plurality of information from a plurality of patients.

20. The information system of claim 1 wherein the specimen-linked database comprises a plurality of information relating to at least one non-living patient.

21. The information system of claim 1 wherein the specimen-linked database comprises a plurality of information relating to at least one living patient.

22. The information system of claim 1 wherein a plurality of tissues or a plurality of cells from the plurality of patients are arrayed on the tissue microarrray.

23. The information system of claim 1 wherein the information system further comprises a plurality of records relating to a plurality of behavioral responses of a plurality of patients having a plurality of neuropsychiatric disorders.

24. The information system of claim 23 wherein the plurality of behavioral responses comprises a plurality of responses to at least one questionnaire.

25. The information system of claim 23 wherein the plurality of behavioral responses are obtained from a plurality of records of psychological evaluations of a plurality of patients.

26. The information system of claim 1 wherein the plurality of information comprises a plurality of information relating to an exposure of at least one patient to at least one drug.

27. An information system comprising:
a tissue microarray having a plurality of samples wherein the plurality of samples are from at least one patient with a neuropsychiatric disorder;
a specimen-linked database comprising information about the tissue microarray wherein the tissue microarray is identified by an identifier;
at least one user device connectable to a network, for displaying an interface wherein a plurality if information can be inputted into the interface; and
an information management system for searching the specimen-linked database and determining relationships between the plurality of information.

28. The information system of claim 27 wherein the information management system displays the plurality of information on the interface of the user device.

29. The information system of claim 27 wherein the information system is stored within at least one server and the information management system is accessible remotely through the network.

30. The information system of claim 27 wherein the information management system is accessible through a readible medium.

31. The information system of claim 27 wherein the information management system is capable of understanding a set of natural query terms.

32. The information system of claim 27 wherein the information management system is capable of understanding Boolean operators and truncation symbols.

33. The information system of claim 27 wherein the information management system generates a plurality of search data from a plurality of terms inputted into the interface of the user device and transfers the plurality of search data to an at least one search engine to initiate a search.

34. The information system of claim 27 wherein the information management system generates a plurality of search data through a selection of options which are displayed on the interface.

35. The information system of claim 27 wherein the information management system is capable of mapping a plurality of data points obtained from the specimen-linked database.

36. The information system of claim 27 wherein the information management system is capable of classifying a plurality of tissue information by a type or an attribute.

37. The information system of claim 27 wherein the information management system is capable of assigning a relationship identification number to each of a plurality of the attributes and storing the relationship identification numbers in the specimen-linked database where the attributes are indexed by the relationship identification number and provided with a descriptor.

38. The information system of claim 27 wherein the information management system comprises a statistical program to identify a plurality of attributes as representing a particular relationship.

39. The information system of claim 27 wherein the information management system is capable of analyzing a particular relationship between a plurality of data in the specimen-linked database using any of the methods in the group consisting of regression, decision trees, neural networks, and fuzzy logic.

40. The information system of claim 27 wherein the information management system further comprises an expert system.

41. The information system of claim 40 wherein the expert system further comprises a transaction manager wherein the transaction manager directs and outputs requests between an at least one server of the information system and an at least one interface of an at least one user device of the information system.

42. The information system of claim 27 wherein the specimen-linked database comprises a plurality of information relating to at least one treatment.

43. The information system of claim 27 wherein the specimen-linked database further comprises a physiological response database wherein the physiological response database includes a plurality of information relating to an at least one physiological response of the patient with the neuropsychiatric disorder.

44. The information system of claim 43 wherein the physiological response database comprises a plurality of information relating to the expression of at least one α₂ adrenoreceptors.

45. The information system of claim 43 wherein the physiological response database comprises a plurality of information relating to at least one β-adrenoreceptor pathway molecule.

46. The information system of claim 43 wherein the physiological response database comprises a plurality of information relating to an expression of at least one dopamine receptor pathway molecule.

47. The information system of claim 43 wherein the physiological response database comprises a plurality of information relating to an opioid receptor pathway molecule.

48. The information system of claim 43 wherein the physiological response database comprises a plurality of information relating to at least one cannabinoid pathway molecule.

49. The information system of claim 43 wherein the physiological response database comprises a plurality of information relating to at least one muscarinic receptor pathway molecule.

50. The information system of claim 43 wherein the physiological response database comprises a plurality of information relating to at least one AMPA receptor pathway molecule.

51. The information system of claim 43 wherein the physiological response database comprises a plurality of information relating to at least one metabotrobic glutamate receptor pathway molecule.

52. The information system of claim 43 wherein the physiological response database comprises a plurality of information relating to at least one serotonin receptor pathway molecule.

53. The information system of claim 43 wherein the physiological response database comprises a plurality of information relating to at least one nicotinic receptor pathway molecule.

54. A method for obtaining information relating to physiological responses of a patient suspected of having a neuropsychiatric disorder comprising:
creating a tissue microarray wherein the tissue microarray includes a plurality of sublocations and a plurality of samples wherein each sublocation includes a sample;
identifying the tissue microarray with an identifier and identifying each sublocation with a set of coordinates;
treating each of the plurality of samples with a molecular probe;
entering a plurality of information relating to each sample into a specimen-linked database; and
correlating the identifier of the tissue microarray and the coordinates of the sublocation with the plurality of information using an information management system.

55. The method of claim 54 further comprising a plurality of tissure microarrays.

56. The method of claim 55 wherein at least one tissue microarray is a control tissue microarray.

57. The method of claim 54 wherein the information management system is capable of searching the specimen-linked database and determining a plurality of relationships between a plurality of information.

58. The method of claim 54 wherein the plurality of information relates to an expression of at least one neurotransmittor receptor pathway biomolecule.

59. The method of claim 54 wherein the plurality of information relates to a pluraltiy of behavioral responses of at least one patient.

60. The method of claim 54 wherein the plurality of information relates to an expression of an at least one neurally expressed gene in at least one sample on the tissue microarray.

61. The method of claim 54 wherein the plurality of information relates to the expression of at least one EST sequence in a sample on the tissue microarray.

62. The method of claim 54 wherein the molecular probe is selected from the group consisting of a nucleic acid, an aptamer, an antibody, or combinations thereof.

63. The method of claim 54 wherein the information management system identifies a plurality of relationships between an at least one expression of a biomolecule and an at least one neuropsychiatric disease.

64. The method according to claim 63 wherein the plurality of relationships identified is used to provide a diagnosis.

65. A method for obtaining information about a sample in a tissue microarray comprising:
creating the tissue microarray comprising a plurality of samples wherein at least one of the samples being from a patient with a neuropsychiatric disorder;
treating each of the plurality of samples with a molecular probe;
entering a plurality of information relating to each sample into a specimen-linked database;
correlating the identifier of the tissue microarray and the coordinates of a sublocation with the plurality of information using an information management system; and
searching the specimen-linked database with the information management system and displaying a plurality of search results on an interface of a user device.

66. The method of claim 65 further comprising comparing a first plurality of information, wherein the first plurality of information has been incorporated into the specimen-linked database, with a second plurality of information, wherein the second plurality of information has not been incorporated into the specimen-linked database.

67. The method of claim 66 wherein the information management system is capable of determining a plurality of relationships between the first plurality of information and the second plurality of information.

68. The method of claim 67 further comprising displaying the plurality of relationships on the interface of the user device.

69. The method of claim 65 wherein the user device is coupled to a molecular profiling system.

70. The method of claim 65 wherein the specimen-linked database includes a data model wherein the data model organizes a plurality of information.

71. The method of claim 65 wherein the information management system is stored within an at least one server and the information management system is accessible remotely through a network.

72. The method of claim 65 wherein the information management system is capable of understanding a set of natural language query terms.

73. The method of claim 65 wherein the information management system is capable of understanding Boolean operators and truncation symbols.

74. The method of claim 65 wherein the information management system generates a plurality of search data through a selection of options which are displayed on the interface.

75. The method of claim 65 wherein the information management system is capable of mapping a plurality of data points obtained from the specimen-linked database.
